(19) United States
(12) Patent Application Publication
(10) Pub. No.: US 2021/0333232 A1
(43) Pub. Date: Oct. 28, 2021

(54) GAS CONCENTRATION DETECTION DEVICE

(71) Applicant: DENSO CORPORATION, Kariya-City (JP)

(72) Inventors: Toshihiko HARADA, Nisshin-City (JP); Kensuke TAKIZAWA, Nisshin-City (JP); Hiroki ICHIKAWA, Kariya-City (JP)

(21) Appl. No.: 17/370,182

(22) Filed: Jul. 8, 2021

Related U.S. Application Data
(63) Continuation of application No. PCT/JP2019/049087, filed on Dec. 16, 2019.

(30) Foreign Application Priority Data
Jan. 9, 2019 (JP) 2019-001753

(51) Int. Cl. G01N 27/407; G01N 27/416; G01N 27/41; G01N 33/00; G01N 27/409

(52) U.S. Cl. CPC G01N 27/4074; G01N 27/416; G01N 27/409; G01N 33/0037; G01N 33/0054; G01N 27/41

(57) ABSTRACT

A gas concentration detection device has a first element part, a first detection part, a second element part, a second detection part and a sensitivity correction part. The sensitivity correction part is configured to correct a second gas component concentration detected by the second detection part based on a time difference between a first response time of the first detection part and a second response time of the second detection part when the first detection part and the second detection part have a function of detecting a common gas component contained in a target gas to be detected, and a variation in concentration of the common gas component exceeds a reference variation amount.

### CROSS-REFERENCE TO RELATED APPLICATION

The present application is a continuation application of International Application No. PCT/JP2019/049087 filed on Dec. 16, 2019, which is based on and claims the benefit of priority from Japanese Patent Application No. 2019-001753 filed on Jan. 9, 2019. The contents of these applications are incorporated herein by reference in their entirety.

### TECHNICAL FIELD

The present disclosure relates to gas concentration detection devices.

### BACKGROUND

For example, a catalyst is arranged in an exhaust gas pipe of a vehicle in order to purify NOₓ (Nitrogen oxides), e.g. NO, NO₂ contained in an exhaust gas emitted from a diesel engine, etc. as an internal combustion engine. In a selective reduction catalyst (SCR), as one of catalysts, ammonia (NH₃) contained in urea water is adhered on a catalyst support. The catalyst support and ammonia reduce NOₓ., i.e. a chemical reaction occurs between ammonia and NOₓ on the catalyst support so as to reduce NOx to nitrogen (N₂) and water (O₂).

A reductant supply device for supplying ammonia as a reductant to a selective reduction catalyst is also arranged at an upstream side of the flow of an exhaust gas in an exhaust gas pipe. For example, a NOₓ sensor and an ammonia sensor are arranged at a downstream side of the selective reduction catalyst in the flow of the exhaust gas in the exhaust gas pipe so as to detect a NOₓ concentration and an ammonia concentration in the exhaust gas. This reduces a leakage of ammonia from the selective reduction catalyst, and increases a purification rate of NOₓ by ammonia on the basis of a NOₓ amount and an ammonia amount detected by the NOₓ sensor and the ammonia sensor.

A multi-component gas sensor is provided with a NOₓ sensor part and an ammonia sensor part which are assembled together in one body. The NOₓ sensor part detects a NOₓ concentration in a target gas to be detected. The ammonia sensor part detects an ammonia concentration in the target gas. The ammonia sensor part is arranged on an outer surface of the NOₓ sensor part so as to be adequately in contact with the target gas.

In a gas concentration detection device, an adequate flow amount of the target gas to be detected is supplied to an electrode of the ammonia detection part in order to detect a presence of ammonia contained in the target gas. This can enhance a detection sensitivity of the ammonia detection part. A solid electrolyte and the electrode of the ammonia detection part are accordingly arranged on one outer surface of a sensor element having a parallelepiped shape. Further, such a sensor element in a sensor main body is supported by a housing casing in which a male screw part of the housing casing is engaged with a female screw part of a mounting part of an exhaust gas pipe. This mounts the sensor main body in the gas concentration detection device in the exhaust gas pile.

However, when the sensor main body having the sensor element and a sensor housing casing is mounted in the exhaust gas pipe, the sensor main body is arranged to be rotated around the mounting part of the exhaust gas pipe. It is accordingly difficult to determine an arrangement direction of the outer surface of the sensor element having the solid electrolyte and the electrode in the exhaust gas pipe. It has been found that a difference may occur in detection sensitivity of the ammonia detection part between two locations, in which the outer surface of the sensor element is arranged toward the upstream side of the target gas flow in the exhaust gas pipe and the outer surface of the sensor element is arranged toward the downstream side of the target gas flow in the exhaust gas pipe.

### SUMMARY

It is desired for the present disclosure to provide a gas concentration detection device having a first element part, a first detection part, a second element part, a second detection part and a sensitivity correction part. The first element part includes a first solid electrolyte body having ionic conductivity, a pair of first electrodes arranged on the first solid electrolyte body, and, a diffusion resistance part and a gas chamber. A target gas to be detected is introduced into the gas chamber through the diffusion resistance part. The first detection part detects a first gas concentration contained in the target gas on the bases of a direct current flowing between the pair of first electrodes when a direct current voltage is applied to the pair of first electrodes and a flow amount of the target gas into the gas chamber is adjusted by the diffusion resistance part. The second element part includes a second solid electrolyte body having ionic conductivity and a pair of second electrodes. The second solid electrolyte body is laminated on the first electrolyte body through an insulator. The second detection part detects a second gas component concentration contained in the target gas on the basis of a potential difference generated between the pair of second electrodes when at least one of the pair of second electrodes is arranged on an outer surface of the second electrolyte body exposed to the target gas to be detected. When the first detection part and the second detection part detect a variation in concentration of a common gas component, which is more than a reference variation amount, contained in the target gas, the sensitivity correction part corrects the second gas component concentration detected by the second detection part based on an output variation time difference and a response time difference or a response speed difference.

### BRIEF DESCRIPTION OF THE DRAWINGS

A preferred, non-limiting embodiment of the present disclosure will be described by way of example with reference to the accompanying drawings, in which:

FIG. 1 is a view showing a cross section explaining a structure of a gas concentration detection device according to a first embodiment of the present disclosure;

FIG. 2 is a view showing a cross section of a sensor element according to the first embodiment along the line II-II shown in FIG. 1;

FIG. 3 is a view showing a cross section of the sensor element according to the first embodiment along the line III-III shown in FIG. 1;

FIG. 4 is a view showing a cross section of the sensor element according to the first embodiment along the line IV-IV shown in FIG. 1;

FIG. 5 is a view explaining an electrical structure of a sensor control unit according to the first embodiment;

FIG. 6 is a view explaining an arrangement of the gas sensor according to the first embodiment in an internal combustion engine;

FIG. 7 is a view explaining an arrangement of a sensor main body in the gas concentration detection device in an exhaust gas pipe of the internal combustion engine;

FIG. 8 is a view explaining a mixed electric potential generated at a detection electrode according to the first embodiment;

FIG. 9 is a view explaining a mixed electric potential generated at the detection electrode according to the first embodiment when an ammonia concentration varies;

FIG. 10 is a view explaining a mixed electric potential generated at the detection electrode according to the first embodiment when an oxygen concentration varies;

FIG. 11 is a graph showing a relationship between an ammonia concentration and an electric potential difference when an oxygen concentration varies according to the first embodiment;

FIG. 12 is a graph showing a relationship between an electric potential difference and an ammonia concentration after performing a correction of an oxygen concentration when an oxygen concentration varies according to the first embodiment;

FIG. 13 is a graph showing a relationship between an angle of an ammonia electrode surface to an upstream side of a flow of an exhaust gas and each sensor output of a first detection part and a second detection part according to the first embodiment;

FIG. 14a is a view explaining an arrangement of the sensor element when the angle of the ammonia electrode surface toward the upstream side of the flow of the exhaust gas is 90° according to the first embodiment;

FIG. 14b is a view explaining an arrangement of the sensor element when the angle of the ammonia electrode surface toward the upstream side of the flow of the exhaust gas is 135° according to the first embodiment;

FIG. 14c is a view explaining an arrangement of the sensor element when the angle of the ammonia electrode surface toward the upstream side of the flow of the exhaust gas is 180° according to the first embodiment;

FIG. 14d is a view explaining an arrangement of the sensor element when the angle of the ammonia electrode surface toward the upstream side of the flow of the exhaust gas is 225° according to the first embodiment;

FIG. 14e is a view explaining an arrangement of the sensor element when the angle of the ammonia electrode surface toward the upstream side of the flow of the exhaust gas is 270° according to the first embodiment;

FIG. 15 is a graph showing a relationship between an angle of the ammonia electrode surface toward the upstream side of the flow of the exhaust gas and a response time of each of the first detection part and the second detection part according to the first embodiment;

FIG. 16 is a graph showing a method of calculating a response time of each of the first detection part and the second detection part according to the first embodiment when a sensor output of each of the first detection part and the second detection part is increased;

FIG. 17 is a graph showing a method of calculating a response time of each of the first detection part and the second detection part according to the first embodiment when a sensor output of each of the first detection part and the second detection part is reduced;

FIG. 18 is a graph showing a method of correcting the sensor output of each of the first detection part and the second detection part according to the first embodiment;

FIG. 19 is a graph showing a method of calculating a variation of the sensor output of each of the first detection part and the second detection part according to the first embodiment when the sensor output of each of the first detection part and the second detection part is increased;

FIG. 20 is a graph showing a method of calculating each response speed according to the first embodiment when a sensor output of each of the first detection part and the second detection part is increased;

FIG. 21 is a flow chart explaining a control method of the gas concentration detection device according to the first embodiment;

FIG. 22 is a view explaining an electrical structure of a sensor control unit according to a second embodiment;

FIG. 23 is a graph showing a relationship between an angle of the ammonia electrode surface toward the upstream side of the flow of the exhaust gas and a response time of each of the first detection part and the second detection part according to the second embodiment;

FIG. 24 is a graph showing a method of correcting the sensor output of each of the first detection part and the second detection part according to the second embodiment;

FIG. 25 is a view explaining an electrical structure of a sensor control unit according to a third embodiment;

FIG. 26 is a flow chart explaining a control method of the gas concentration detection device according to the third embodiment;

FIG. 27 is a flow chart explaining a control method of the gas concentration detection device according to the third embodiment;

FIG. 28 is a view showing a cross section explaining a structure of the gas concentration detection device according to the fourth embodiment of the present disclosure; and

FIG. 29 is a view explaining an electrical structure of a sensor control unit according to the fourth exemplary embodiment.

### DETAILED DESCRIPTION OF THE PREFERRED EMBODIMENTS

Hereinafter, various embodiments of the present disclosure will be described with reference to the accompanying drawings. In the following description of the various embodiments, like reference characters or numerals designate like or equivalent component parts throughout the several diagrams.

Next, a description will be given of a gas concentration detection device according to preferred embodiments of the present disclosure.

### First Embodiment

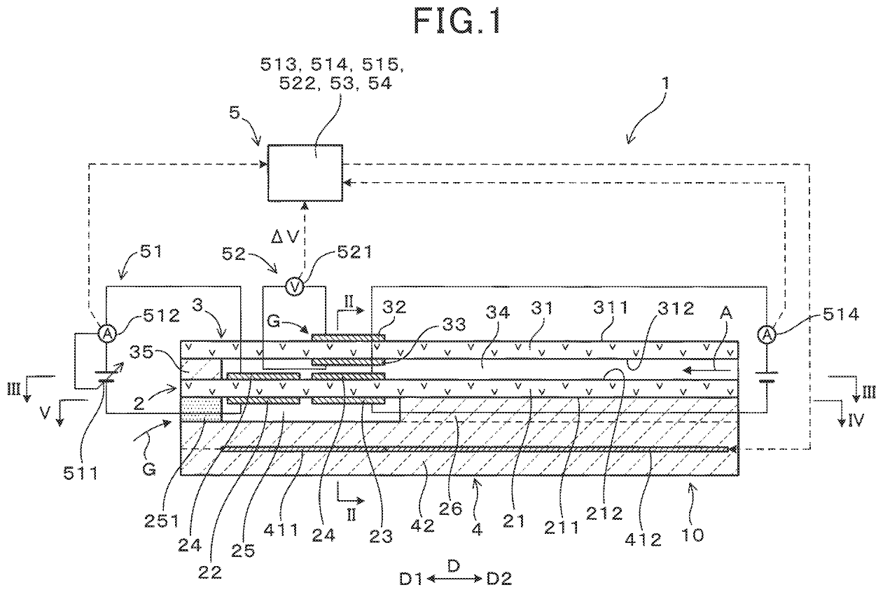

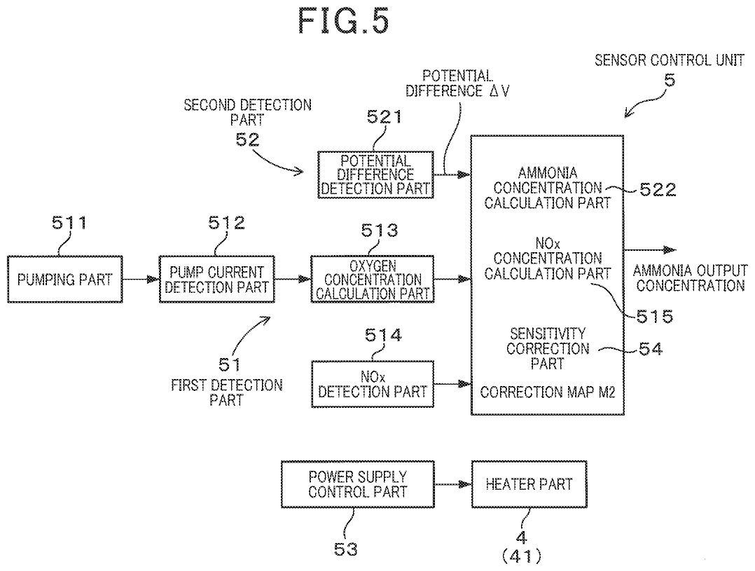

As shown in FIG. 1 and FIG. 5, the gas concentration detection device 1 according to the first embodiment includes a first element part (oxygen element part) 2, a first detection part 51, a second element part (ammonia element part) 3, a second detection part 52 and a sensitivity correction part 54. The first element part 2 has a first solid electrolyte body 21 having ionic conductivity, a pair of first electrodes 22 and 24 and a gas chamber 25. The pair of first electrodes 22 and 24 are formed on the first solid electrolyte body 21. The gas chamber 25 accommodates a target gas G to be detected. The target gas G is introduced into the gas chamber 25 through a diffusion resistance part 251. The gas chamber 25 accommodates one of the pair of first electrodes 22 and 24. The first detection part 51 detects a first gas component concentration in the target gas G to be detected on the basis of a limiting current flowing between the pair of first electrodes 22 and 24 when the diffusion resistance part 251 adjusts a flow amount of the target gas G into the gas chamber 25, and a direct current voltage is applied between the pair of first electrodes 22 and 24.

The second element part 3 has a second solid electrolyte body 31 having ionic conductivity and a pair of second electrodes 32 and 33. The second solid electrolyte body 31 is stacked on the first solid electrolyte body 21 through a duct insulator 35. The pair of second electrodes 32 and 33 are formed on the second solid electrolyte body 31. In a situation in which one of the pair of second electrodes 32 is formed on an outer surface of the second solid electrolyte body 31, which is exposed to the target gas G to be detected, the second detection part 52 is configured to detect a second gas component concentration in the target gas G on the basis of an electrical potential difference ΔV generated between the pair of second electrodes 32 and 33.

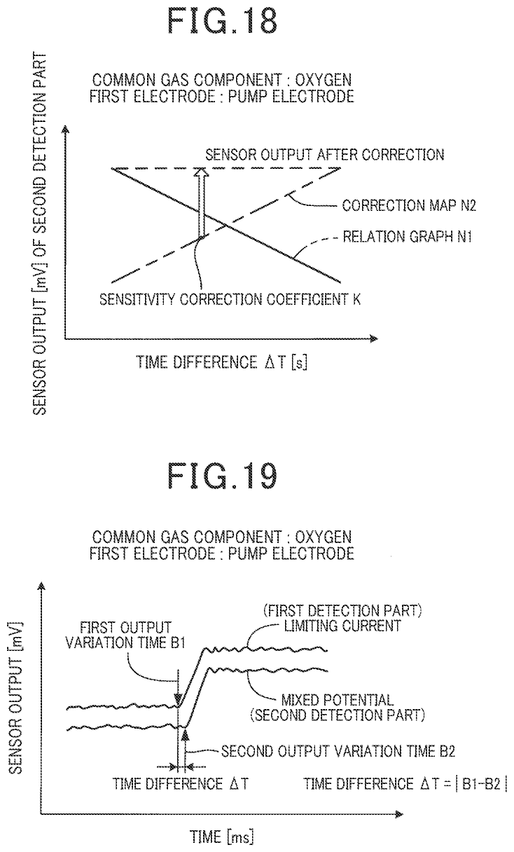

As shown in FIG. 15 and FIG. 18, the sensitivity correction part 54 is configured to correct a second gas component concentration detected by the second detection part 52 on the basis of a time difference ΔT between the first response time T1 of the first detection part 51 and the second response time T2 of the second detection part 52 when both the first detection part 51 and the second detection part 52 detect occurrence of a variation of a common gas component contained in the target gas G to be detected, which is not less than a reference variation amount. Both the first detection part 51 and the second detection part 52 have a detection sensitivity to the common gas component contained in the target gas G to be detected.

Hereinafter, a description will be given of the gas concentration detection device 1 according to the present embodiment.

### (Gas Concentration Detection Device 1)

As shown in FIG. 1, the gas concentration detection device 1 according to the present embodiment is configured to detect gas component concentrations of not less than two types contained in the target gas G to be detected. The gas concentration detection device 1 is a multi-component gas sensor. The present embodiment detects an exhaust gas emitted from an internal combustion engine 7 as the target gas G containing NOₓ (nitrogen oxides) as a first gas component and ammonia (NH₃) as a second gas component.

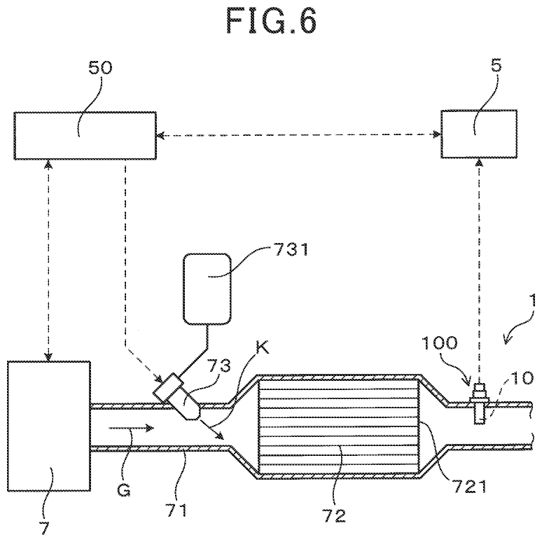

As shown in FIG. 6, the gas concentration detection device 1 detects a NOₓ concentration and an ammonia concentration flowing through a catalyst 72 for reducing NOₓ in the exhaust gas pipe 71 of the internal combustion engine (engine) 7 of a vehicle. The target gas G to be detected is an exhaust gas emitted from the internal combustion engine 7 and flows in the exhaust gas pipe 71. A chemical composition of the exhaust gas varies according to a combustion state in the internal combustion engine 7. In a fuel rich situation in which an air-fuel ratio in the internal combustion engine 7 is higher than the theoretical air-fuel ratio, an amount of HC (hydrocarbon), CO (carbon monoxide), H₂ (hydrogen), etc. contained in un-combusted gas is increased, and on the other hand, an amount of NOₓ (nitrogen oxide), e.g. NO, NO₂, N₂O, etc. is reduced. In a fuel lean situation in which an air-fuel ratio in the internal combustion engine 7 is lower than the theoretical air-fuel ratio, an amount of HC, CO, etc. in a chemical composition of an exhaust gas is reduced, and an amount of NOₓ is increased. In addition, in a fuel rich situation, oxygen (air) is substantially absent in the target gas G to be detected. In a fuel lean situation, more oxygen (air) is contained in the target gas G to be detected.

### (Catalyst 72)

As shown in FIG. 6, the catalyst 72 and a reductant supply device 73 are arranged in the exhaust gas pipe 71. The catalyst 72 reduces NOₓ, and the reductant supply device 73 supplies a reductant K containing ammonia to the catalyst 72. The catalyst 72 includes catalyst supports on which ammonia as the reductant K is adhered. An amount of ammonia on the catalyst supports in the catalyst 72 is reduced with progress of its reduction reaction. When an amount of ammonia adhered on the catalyst supports is less, the reductant supply device 73 supplies additional ammonia to the catalyst supports. The reductant supply device 73 is arranged at a location in the upstream side of the flow of the exhaust gas when compared with a location of the catalyst 72 in the exhaust gas pipe 71. When the reductant supply device 73 sprays ammonia, ammonia gas is generated in the exhaust gas pipe 71. Urea water is hydrolyzed to generate ammonia gas. A urea water tank 731 is joined to the reductant supply device 73.

The internal combustion engine 7 used in the present embodiment is a diesel engine performing combustion utilizing the self-ignition of light oil. The catalyst 72 is a selective catalytic reduction (SCR) performing a chemical reaction of NOₓ (nitrogen oxide) and ammonia (NH₃) so as to generate nitrogen (N₂) and water (H₂O).

It is acceptable to arrange an oxidation catalyst (DOC, not shown) and a filter (DPF, not shown) at the upstream side of the catalyst 72 in the exhaust gas pipe 71. The DOC oxidizes NO into NO₂ (oxidation), and decreases an amount of CO, HC (hydrocarbon), etc. in the exhaust gas pipe. The DPF collects particulate matter contained in an exhaust gas.

### (Multi-Component Gas Sensor)

As shown in FIG. 6, the gas concentration detection device 1 according to the present embodiment is arranged at the downstream side of the catalyst 72 in the exhaust gas pipe 71. In more detail, a sensor main body 100 in the gas concentration detection device 1 is arranged in the exhaust gas pipe 71. A sensor control unit (SCU) as a control part of the gas concentration detection device 1 is arranged at an external location of the exhaust gas pipe 71. The present embodiment will also refer to the gas concentration detection device 1 as the sensor main body 100.

The gas concentration detection device 1 according to the present embodiment is configured to detect an ammonia concentration and NOx concentration as a multi-component gas sensor (or a composite sensor). The gas concentration detection device 1 uses an oxygen concentration to correct an ammonia concentration. An engine control unit (ECU) 50 as a control device of the internal combustion engine 7 uses an ammonia concentration and a NOx concentration transmitted from the gas concentration detection device 1 so as to determine a timing to supply ammonia as the reductant K of the reductant supply device 73 to the exhaust gas pipe 71.

The internal combustion engine 7 uses the engine control unit 50, a sensor control unit 5 and various electronic control units. The sensor control unit 5 controls the operation of the gas concentration detection device 1. These control units are composed of various types of computers (processing units).

The engine control unit 50 is configured to instruct the reductant supply device 73 to inject urea water so as to supply ammonia into the catalyst 72 when the gas concentration detection device 1 detects presence of NOₓ contained in the target gas G to be detected. The detection of the presence of NOₓ in the target gas G indicates an insufficient state of ammonia in the catalyst 72. On the other hand, the engine control unit 50 detects that the catalyst 72 contains an excess amount of ammonia when the presence of ammonia in the target gas G is detected. The engine control unit 50 stops the reductant supply device 73 from injecting ammonia in order to stop the supply of ammonia into the catalyst 72. It is preferable to supply a proper amount of ammonia into the catalyst 72 so as to reduce NOₓ.

The ammonia supply control by the engine control unit 50 allows a NOₓ concentration area and an ammonium concentration area, in the target gas G at the downstream side (catalyst outlet 721) of the catalyst 72 and around the gas concentration detection device 1, to have various states such as a reduction state in which NOₓ is properly reduced by ammonia, a NOₓ excess emission state in which a NOₓ emission amount increases, and an excess ammonia emission state in which an ammonia emission amount increases according to elapse of time.

### (The Main Body 100 of the Sensor)

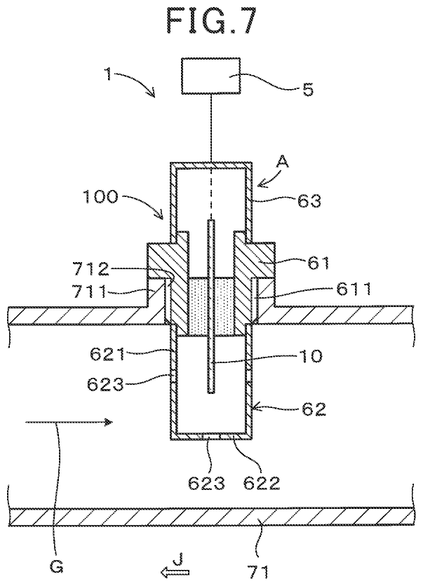

As shown in FIG. 7, the sensor main body 100 of the gas concentration detection device 1 includes a sensor element 10, a housing casing 61, a top-end side cover 62 and a base-end side cover 63. A first element part (oxygen element part) 2, a second element part (ammonia element part) 3, etc. are formed in the sensor element 10. The housing casing 61 is engaged with an attachment part 711 of the exhaust gas pipe 71. The top-end side cover 62 is mounted at the top-end side of the housing casing 61 so as to protect the sensor element 10. The base-end side cover 63 is mounted at the base-end side of the housing casing 61 so as to protect electrical wires of the sensor element 10.

The housing casing 61 includes a male screw part 611 to be engaged with a female screw part 712 of the attachment part 711 of the exhaust gas pipe 71 through which the target gas G to be detected flows. The top-end side cover 62 includes a cylindrical part 621 and a base part 622. The cylindrical part 621 is mounted to the housing casing 61. The base part 622 plugs a top-end part of the cylindrical part 621. Through holes 623 are formed in the cylindrical part 621 and the base part 622, through which the target gas G to be detected flows. The through holes 623 are formed along a circumferential direction of the cylindrical part 621.

FIG. 7 is a schematic view showing the gas concentration detection device 1. It is possible to have various shapes of the top-end side cover 62 and the through holes 623 formed in the top-end side cover 62. For example, it is acceptable for the top-end side cover 62 to have a double cover structure composed of an inside cover and an outside cover arranged at the outer circumferential side of the inside cover.

### (The Sensor Element 100)

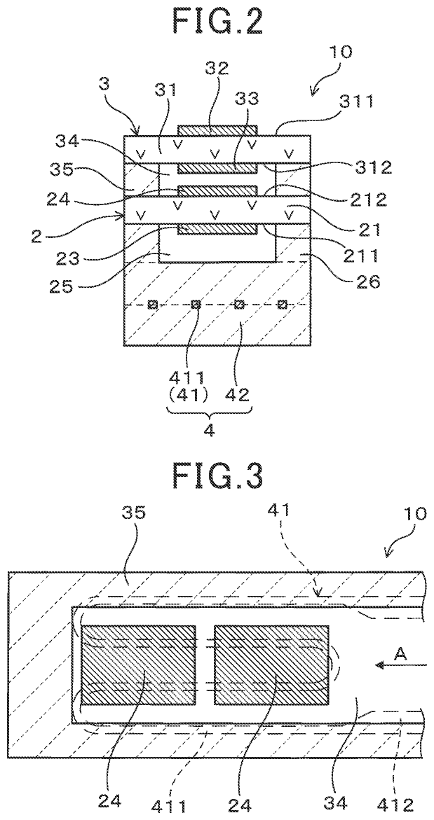

As shown in FIG. 1 and FIG. 2, the sensor element 10 includes the first element part (oxygen element part) 2 and the second element part (ammonia element part) 3. The first element part 2 detects an oxygen concentration and NOₓ concentration. The second element part 3 detects an ammonia concentration. Further, the sensor element 10 includes the first solid electrolyte body 21 and the second solid electrolyte body 31. The first solid electrolyte body 21 forms the first element part 2. The second solid electrolyte body 31 forms the second element part 3. As shown in FIG. 1 to FIG. 3, a heater part 4 is formed in the sensor element 10 so as to heat the first element part 2 and the second element part 3.

The sensor element 10 according to the present embodiment has a rectangular shape having a long side in one direction. A diffusion resistance part 251 is formed at the top-end side part of the sensor element 10 in a longitudinal direction thereof. As shown in FIG. 1, a reference character D indicates the longitudinal direction of the sensor element 10, a reference character D1 indicates the top-end side in the longitudinal direction D, and a reference character D2 indicates the base-end side in the longitudinal direction D. The top-end side of the housing casing 61 and the top-end side D1 in the longitudinal direction D of the sensor element 10 are arranged in the same side.

The first solid electrolyte body 21 and the second solid electrolyte body 31 have a rectangular parallelepiped plate shape. Insulator plates 26, 35 and 42 are stacked on the first solid electrolyte body 21 and the second solid electrolyte body 31. A reference gas duct 34 is formed in the duct insulator plate 35 stacked between the first solid electrolyte body 21 and the second solid electrolyte body 3. A second reference electrode 33 is arranged in the reference gas duct 34. The ammonia electrode 32 is formed on an outer surface 311 of the second solid electrolyte body 31. The outer surface 311 of the second solid electrolyte body 31 is one of outer surfaces (outermost surfaces) of the sensor element 10, into which the target gas G to be detected flows at a predetermined rate.

### (The First Element Part 2 and the Second Element Part 3)

As shown in FIG. 1 to FIG. 4, the pump electrode 22 and the NOₓ electrode 23 are formed on the outer surface 211 of the first solid electrolyte body 21, to which the gas chamber 25 is adjacently arranged. The gas chamber 25 accommodates the pump electrode 22 and the NOₓ electrode 23. The pump electrode 22 adjusts an oxygen concentration in the target gas G to be detected in the gas chamber 25. The NOₓ electrode 23 detects a NOx concentration in the target gas G in the gas chamber 25 after the pump electrode 22 adjusts the oxygen concentration. The reference gas duct 34 and the first reference electrode 24 are formed on the inner surface 212 of the first solid electrolyte body 21, opposite in location to the outer surface adjacently arranged to the gas chamber 25. The reference gas A is introduced into the reference gas duct 34. The reference gas duct 34 accommodates the first reference electrode 24. The pair of first electrodes 22 and 24 are composed of the pump electrode 22 and the first reference electrode 24. The first detection part 51 is configured to detect an oxygen concentration as a first gas component concentration.

The oxygen element part 2 as the first element part 2 is composed of the first solid electrolyte body 21, the pump electrode 22, the NOₓ electrode 23, the first reference electrode 24, the gas chamber 25 and the diffusion resistance part 251. The present embodiment uses, as the pump electrode 22, the first electrode which is exposed to the target gas G in the gas chamber 25, and uses, as the first reference electrode 24, the first electrode arranged in the reference gas duct 34 and exposed to the reference gas A. It is also acceptable to use, as the NOₓ electrode 23, the first electrode exposed to the target gas G in the gas chamber 25.

The ammonia electrode as a mixed potential electrode is formed on the outer surface 311 of the second solid electrolyte body 31. The ammonia electrode detects a mixed potential generated when an electrochemical reduction reaction of oxygen contained in the target gas G and an electrochemical oxidation reaction of ammonia contained in the target gas G are balanced. The second reference electrode 33, accommodated in the reference gas duct 34, is formed on the inner surface 312 of the second solid electrolyte body 31. The second reference electrode 33 is arranged adjacent to the reference gas duct 34 on the inner surface 312.

The ammonia element part 3 as the second element part 3 is composed of the second solid electrolyte body 31, the ammonia electrode 32 and the second reference electrode 33. One of the pair of second electrodes 32 and 33 acts as the ammonia electrode (mixed potential electrode) 32, exposed to the target gas G to be detected. The other of the pair of second electrodes 32 and 33 acts as the second reference electrode 33 arranged in the reference gas duct 34, which is exposed to the reference gas A.

### (The First Detection Part 51 and the Second Detection Part 52)

As shown in FIG. 1 and FIG. 5, the first detection part 51 according to the present embodiment detects an oxygen concentration as the first gas component concentration. The first detection part 51 is composed of a pumping part 511, a pump current detection part 512 and an oxygen concentration calculation part 513. The pumping part 511 will be described later. The second detection part 52 according to the present embodiment detects an ammonia concentration as the second gas component concentration. The second detection part 52 is composed of a potential difference detection part 521 and an ammonia concentration calculation part 522. The potential difference detection part 521 will be described later. The sensitivity correction part 54 according to the present embodiment uses variation in oxygen concentration as the common gas component.

As will be shown in the second embodiment, it is possible to have a structure in which the first detection part 51 detects a NOₓ concentration as the first gas component concentration. In this structure, the first detection part 51 is composed of a NOₓ detection part 514 and a NOₓ concentration calculation part 515 which will be described later.

Oxygen in the target gas G to be detected is discharged through the gas chamber 25 in the pumping electrode 511. The pump current detection part 512 detects a limiting current generated between the pump electrode 22 and the first reference electrode 24. The oxygen concentration calculation part 513 calculates an oxygen concentration on the bases of the limiting current.

The potential difference detection part 521 detects a mixed potential (potential difference ΔV) generated between the ammonia electrode 32 and the second reference electrode 33. The ammonia concentration calculation part 522 calculates an ammonia concentration on the basis of the mixed potential.

### (The Sensor Control Unit 5)

As shown in FIG. 1 and FIG. 5, the first detection part 51 and the second detection part 52 are formed in the sensor control unit in the gas concentration detection device 1. The sensor control unit 5 has the sensitivity correction part 54 which corrects an error of the potential difference ΔV or the second gas component concentration. The potential difference ΔV is generated due to a sensitivity shift of ammonia detected by the second detection part 52. The sensor control unit 5 has a power supply control part 53 which supplies electric power to a heater 41 forming the heater part 4.

### (Detailed Explanation of the Ammonia Element Part 3 as the Second Element Part 3)

As shown in FIG. 1 and FIG. 2, the ammonia element part 3 as the second element part 3 is a part of the sensor element 10 which detects an ammonia concentration. The ammonia element part 3 is composed of the second solid electrolyte body 31 having oxygen ion conductivity, the ammonia electrode 32 and the second reference electrode 33. The ammonia electrode 32 is formed on the outer surface of the second solid electrolyte body 31 and exposed to the target gas G to be detected. The second reference electrode 33 is formed on the inner surface 312 of the second solid electrolyte body 31 and exposed to the reference gas A. The pair of second electrodes 32 and 33 are composed of the ammonia electrode 32 and the second reference electrode 33.

The second solid electrolyte body 31 has a plate shape and is made of zirconia material having oxygen ion conductivity performing at a predetermined temperature. The zirconia material may be composed of various materials, e.g. zirconia as a principal material. It is possible to use, as the zirconia material, a rare earth metal oxide e.g. yttria (yttrium oxide) or, to use a stabilized zirconia or a partially stabilized zirconia in which a part of the zirconia is replaced with an alkaline earth metal.

The ammonia electrode 32 is made of noble metal material containing gold (Au) having catalytic activity to ammonia and oxygen. It is possible for the ammonia electrode 32 to have noble metal materials composed of Pt (platinum)-Au (gold) alloy, Pt—Pd (palladium), Au—Pd alloy, etc. The second reference electrode 33 is composed of noble metal material, e.g. platinum (Pt) having catalytic activity to oxygen. It is acceptable for the ammonia electrode 32 and the second reference electrode 33 to contain zirconia material which becomes a common material on being sintered with the second solid electrolyte body 31.

The outer surface 311 of the second solid electrolyte body 31, to be exposed to the target gas G to be detected, forms the outermost surface of the sensor element 10 of the gas concentration detection device 1. The ammonia electrode 32 formed on the outer surface 311 is adequately in contact with the target gas G. No protection layer composed of porous body, etc. is formed on the surface of the ammonia electrode 32 according to the present embodiment. The ammonia electrode 32 is in contact with the target gas G without diffusion and speed limitations. It is possible to form a protection layer on the surface of the ammonia electrode 32, which does not limit the flow rate of the target gas G.

The second reference electrode 32 formed on the inner surface 312 of the second solid electrolyte body 312 is exposed to air as the reference gas A. A reference duct (air duct) 34, into which air is introduced, is formed in the inner surface 312 of the second solid electrolyte body 31.

### (The Potential Detection Part 512 and the Potential Difference ΔV)

As shown in FIG. 1, a potential difference ΔV detected by the potential difference detection part 521 forming the second detection part 52 is a mixed potential generated when an electrochemical reduction reaction (hereinafter, reduction reaction in short) of oxygen contained in the target gas G and an electrochemical oxidation reaction (hereinafter, oxidation reaction in short) of ammonia contained in the target gas G are balanced. The second detection part 52 is a device of a mixed potential type as a potential difference type. The potential difference detection part 521 is configured to detect a potential difference ΔV between the ammonia electrode 32 and the second reference electrode 33 generated when a reduction current of the reduction reaction at the ammonia electrode 32 becomes equal to an oxidation current of the oxidation reaction of ammonia.

The potential difference detection part 521 according to the present embodiment detects a potential difference ΔV between the ammonia electrode 32 and the second reference electrode 33 generated when a mixed potential is generated at the ammonia electrode 32. Oxidation reaction of ammonia and reduction reaction of oxygen occur simultaneously at the ammonia electrode 32 when ammonia and oxygen are present in the target gas G to be detected which is in contact with the ammonia electrode 32. It is possible to represent the oxidation reaction of ammonia as 2NH₃+3O²⁻→N₂+3H₂O+6e⁻. It is also possible to represent the reduction reaction of oxygen as O₂+4e⁻=→2O²⁻. A mixed potential of ammonia and oxygen at the ammonia electrode 32 is generated as an electric potential when the oxidation reaction (rate) of ammonia becomes equal to the reduction reaction (rate) of oxygen.

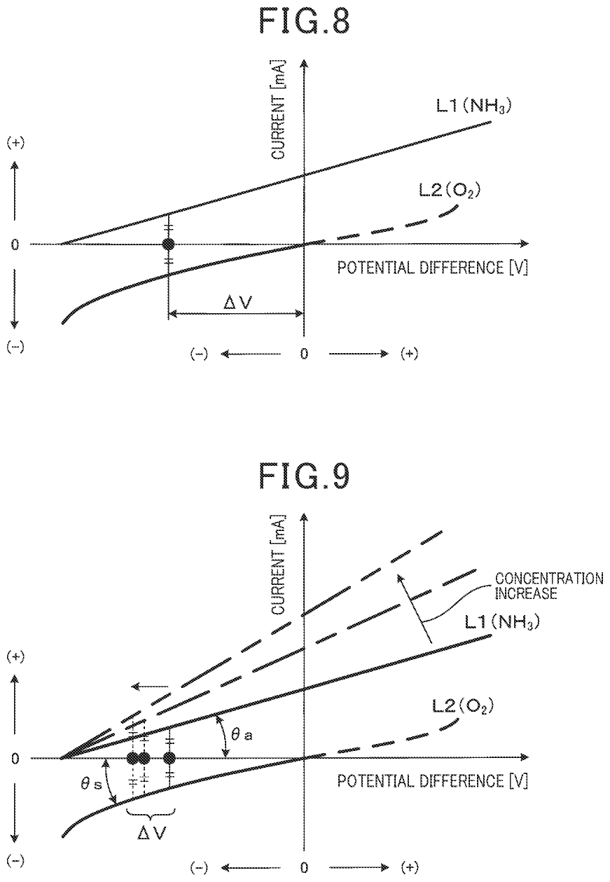

FIG. 8 is a view explaining a mixed potential generated at the ammonia electrode 32. The lateral axis in FIG. 8 represents an electric potential (potential difference ΔV) of the ammonia electrode 32 to the second reference electrode 33, and the vertical axis represents a current flowing between the ammonia electrode 32 and the second reference electrode 33 so as to show a variation of the mixed potential. A first line L1 in FIG. 8 represents a relationship between an electric potential and a current when the oxidation reaction of ammonia occurs at the ammonia electrode 32. A second line L2 in FIG. 8 represents a relationship between an electric potential and a current when the reduction reaction of oxygen occurs at the ammonia electrode 32. In FIG. 8, the first line L1 and the second line L2 are rising steadily.

The potential difference ΔV of 0 (zero) represents that the electric potential of the ammonia electrode 32 is equal to the electric potential of the second reference electrode 33. A mixed potential is an electric potential when a current at a positive side on the first line L1 representing the oxidation reaction of ammonia is balanced with a current at a negative side of the second line L2 representing the reduction reaction of oxygen. The mixed potential at the ammonia electrode 32 is detected as an electric potential at a negative side to the second reference electrode 33.

As shown in FIG. 9, the first line L1 representing the oxidation reaction of ammonia has a large slope θa when the target gas G to be detected has a high ammonia concentration. In this case, when the current at the positive side on the first line L1 is balanced with the current at the negative side on the second line L2, the electric potential becomes shifted toward the negative side. Accordingly, the higher the ammonia concentration becomes, the more the electric potential at the ammonia electrode 32 is shifted toward the negative side, relative to the second reference electrode 33 shifted toward the negative side of the ammonia electrode 32. In other words, the higher the ammonia concentration is, the greater the potential difference (mixed potential) ΔV between the ammonia electrode 32 and the second reference electrode 33. That is, the higher the ammonia concentration, the greater the potential difference ΔV becomes. It is accordingly possible to detect an ammonia concentration in the target gas G on the basis of the detection of the potential difference ΔV.

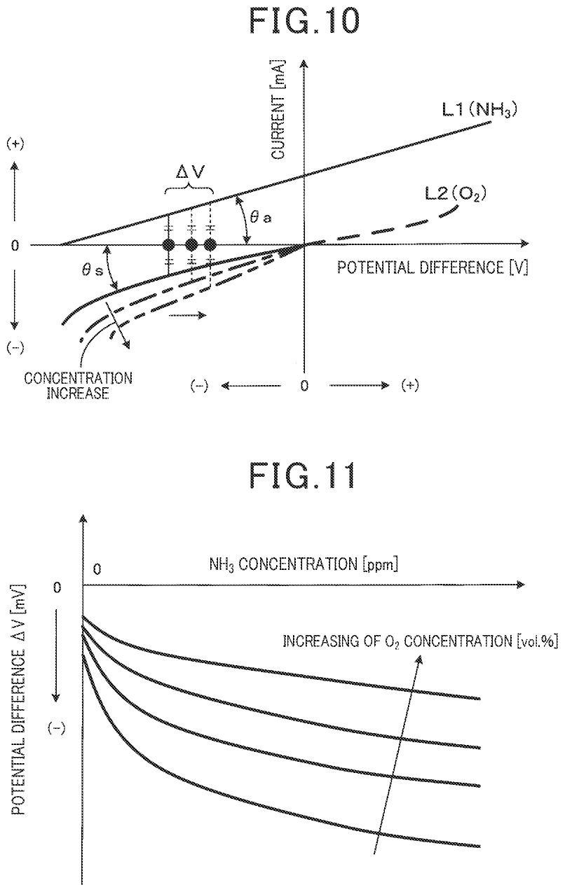

As shown in FIG. 10, when the oxygen concentration in the target gas G becomes high, the second line L2 representing the reduction reaction of oxygen has a large slope θs. In this case, when the current at the positive side on the first line L1 is balanced with the current at the negative side on the second line L2, the electric potential which is shifted toward zero on the negative side. Accordingly, the higher the oxygen concentration becomes, the more the electric potential at the negative side of the ammonia electrode 32 to the second reference electrode 33 is reduced. In other words, the higher the oxygen concentration is, the smaller the potential difference (mixed potential) ΔV between the ammonia electrode 32 and the second reference electrode 33 becomes. The more the oxygen concentration becomes high, it is accordingly possible to enhance the detection accuracy of the ammonia concentration by performing the correction of the electric potential ΔV or the ammonia concentration.

### (Detailed Explanation of the Oxygen Element Part 2 as the First Element Part 2)

As shown in FIG. 1 and FIG. 2, the oxygen element part 2 as the first element part 2 is a part of the sensor element 10 to detect an oxygen concentration and a NOₓ concentration. The oxygen element part 2 includes the first solid electrolyte body 21 having oxygen ionic conductivity, the pump electrode 22 and the NOₓ electrode 23, and the first reference electrode 24. The pump electrode 22 and the NOx electrode 23 are formed on the outer surface 211 of the first solid electrolyte body 21 and exposed to the target gas G to be detected. The first reference electrode 24 is formed on the inner surface 212 of the first solid electrolyte body 21 and exposed to the reference gas A. The pair of first electrodes 22 and 24 are composed of the pump electrode 22 and the first reference electrode 24. The oxygen element part 2 has the gas chamber 25 in which the pump electrode 22 and the NOₓ electrode 23 are accommodated. The target gas G to be detected is introduced into the gas chamber 25 through the diffusion resistance part 25.

The first solid electrolyte body 21 is arranged facing the second solid electrolyte body 31 through the reference gas duct 34. The first solid electrolyte body 21 has a plate shape and is made of zirconia material having oxygen ionic conductivity at a predetermined temperature. This zirconia material is the same as the second solid electrolyte body 31.

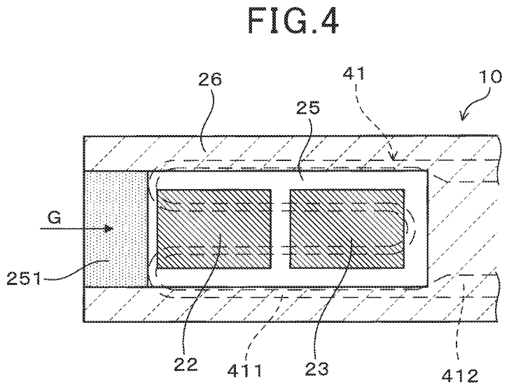

As shown in FIG. 1, FIG. 2 and FIG. 4, the gas chamber 25 is formed in contact with the outer surface 211 of the first solid electrolyte body 21. The gas chamber 25 is formed by using a gas chamber insulator 26. The gas chamber insulator 26 is made of ceramic material, e.g. alumina, etc. The diffusion resistance part 251 is formed as a porous ceramic layer, through which the target gas G to be detected is introduced. The diffusion resistance part 251 limits a diffusion rate of the target gas G.

The pump electrode 22 is formed on the outer surface 211 of the first solid electrolyte body 21. The pump electrode 22 is accommodated in the gas chamber 25, and exposed to the target gas G. The NOₓ electrode 23 is formed on the outer surface 211 of the first solid electrolyte body 21. The NOₓ electrode 23 is accommodated in the gas chamber 25, and exposed to the target gas G after the pump electrode 22 adjusts the oxygen concentration. The first reference electrode 24 is formed on the inner surface 212 of the first solid electrolyte body 21, which is formed opposite to the outer surface 211.

The pump electrode 22 is made of noble metal material having catalytic activity to oxygen, and no activity to NOₓ. It is possible to form the pump electrode 22 by using noble metal material, e.g. a Pt—Au alloy, or a material containing Pt or Au. The NOₓ electrode 23 is made of a noble metal material having catalytic activity to NOₓ and oxygen. It is possible to form the NOx electrode 23 by using a noble metal material containing a Pt—Rh (rhodium) alloy, or a material containing Pt or Rh. The first reference electrode 24 is formed by using a noble metal material, e.g. Pt having catalytic activity to oxygen. It is acceptable for the pump electrode 22, the NOₓ electrode 23 and the first reference electrode 24 to contain zirconia material as a common material on being sintered with the first solid electrolyte body 21.

The first reference electrode 24 according to the present embodiment is formed opposite to the location of the pump electrode 22 through the first solid electrolyte body 21 and on the location opposite to the location of the NOₓ electrode 23 through the first solid electrolyte body 21. It is acceptable to form the first reference electrode 24 on the entire area of one of the locations opposite to the pump electrode 22 and the location opposite to the NOₓ electrode 23.

As shown in FIG. 1 to FIG. 3, the first reference electrode 24 formed on the inner surface 212 of the first solid electrolyte body 21 is exposed to air as the reference gas A. The first solid electrolyte body 21 and the second solid electrolyte body 31 are stacked through the duct insulator plate 35 which forms the reference gas duct 34. The duct insulator plate 35 is made of ceramic material, e.g. alumina, etc.

The reference gas duct 34 is formed to have a structure in which the first reference electrode 24 on the inner surface 212 of the first solid electrolyte body 21 and the second reference electrode 33 formed on the inner surface 312 of the second solid electrolyte body 31 are exposed to air. The first reference electrode 24 and the second reference electrode 33 are accommodated in the reference gas duct 34. The reference gas duct 34 is formed from a base-end side of the sensor element 10 to a location opposite to the location of the gas chamber 25.

As shown in FIG. 7, the reference gas A is introduced into the inside of the base-end side cover 63 through opening holes formed in the base-end side cover 63 of the sensor body 100. The reference gas A is further introduced into the inside of the reference gas duct 34 through the opening part at the base-end side of the reference gas duct 34. The sensor element 10 according to the present embodiment has the reference gas duct 34 formed between the first solid electrolyte body 21 and the second solid electrolyte body 31. This structure makes it possible to allow the entirely of the first reference electrode 24 and the second reference electrode 33 to be exposed to air.

### (The Pumping Part 511, the Pump Current Detection Part 512 and the Oxygen Concentration Calculation Part 513)

As shown in FIG. 1 and FIG. 5, the pumping part 511 is formed to extract oxygen from the target gas G in the gas chamber 25 on supplying a direct current between the pump electrode 22 and the first reference electrode 24. When a direct current is supplied between the pump electrode 22 and the first reference electrode 24, oxygen ions from oxygen contained in the target gas G in the gas chamber 25 migrate to the first reference electrode 24 through the first solid electrolyte body 21, and are discharged from the first reference electrode 24 to the reference gas duct 34. This adjusts an oxygen concentration in the gas chamber 25 into its necessary concentration so as to detect NOₓ.

The pump current detection part 512 is configured to detect a limiting current as a direct current (pump current) which flows between the pump electrode 22 and the first reference electrode 24 when the pumping part 511 provides a direct current between the pump electrode 22 and the first reference electrode 24. The oxygen concentration calculation part 513 is configured to detect an oxygen concentration in the target gas G on the basis of a limiting current detected by the pump current detection part 512. The pump current detection part 512 detects a direct current which is in proportion to an oxygen amount discharged to the reference gas duct 34 from the gas chamber 25 by the pumping part 511.

The pumping part 511 discharges oxygen in the gas chamber 25 to the reference gas duct 34 until the oxygen concentration in the target gas G in the gas chamber 25 is changed to a predetermined concentration. Accordingly, it is possible for the oxygen concentration calculation part 513 to calculate an oxygen concentration in the target gas G reaching the oxygen element part 2 and the ammonia element part 3 while monitoring the limiting current detected by the pump current detection part 512.

The ammonia concentration calculation part 522 corrects an ammonia concentration on the basis of the oxygen concentration calculated by the oxygen concentration calculation part 513.

(The NOₓ Detection Part 514 and the NOₓ Concentration Calculation Part 515)

As shown in FIG. 1 and FIG. 5, the NOₓ detection part 514 is configured to detect a direct current (sensor current) flowing between the NOₓ electrode 23 and the first reference electrode 24 when supplying a direct current between the NOₓ electrode 23 and the first reference electrode 24 as a positive side. The NOₓ concentration calculation part 515 calculates an uncorrected NOₓ concentration in the target gas G based on the limiting current detected by the NOₓ detection part 514. The NOₓ concentration calculation part 515 calculates a corrected NOₓ concentration obtained by subtracting an ammonia concentration detected by the ammonia concentration calculation part 522 from an uncorrected NOₓ concentration. The NOₓ detection part 514 detects the presence of ammonia in addition to NOₓ. The NOₓ concentration calculation part 515 obtains an actual detection amount of NOₓ by subtracting the detected amount of ammonia from the detected amount of NOₓ.

The NOₓ concentration calculation part 515 provides two types of NOₓ concentration. The NOₓ concentration calculated based on a current generated in the NOₓ detection part 514 is an uncorrected NOₓ concentration. The uncorrected NOₓ concentration contains an ammonia concentration of ammonia reacted at the NOₓ electrode 23. On the other hand, the corrected NOₓ concentration is obtained by subtracting the ammonia concentration obtained by the ammonia concentration calculation part 522 from the uncorrected NOₓ concentration obtained by the NOₓ concentration calculation part 515. The corrected NOₓ concentration represents a NOₓ concentration without any influence of ammonia.

The NOₓ electrode 23 is in contact with the target gas G in which an oxygen concentration is adjusted by the pump electrode 22. When a direct current is applied between the NOₓ electrode 23 and the first reference electrode 24, NOₓ in contact with the NOₓ electrode 23 is decomposed to nitrogen and oxygen. Oxygen becomes oxygen ions, and the oxygen ions pass into the first solid electrolyte body 21 to the first reference electrode 24. The oxygen ions are discharged to the reference gas duct 34 from the first reference electrode 24. NOₓ generated by oxidation of ammonia reaches the NOₓ electrode 23. This NOₓ is decomposed to nitrogen and oxygen. The NOₓ concentration calculation part 515 monitors the limiting current detected by the NOₓ detection part 514, and calculates an uncorrected NOₓ concentration in the target gas G reaching the oxygen element part 2. The NOₓ concentration calculation part 515 calculates a corrected NOₓ concentration obtained by subtracting the ammonia concentration from the uncorrected NOₓ concentration.

The gas concentration detection device 1 has a multi-component gas sensor for detecting an ammonia concentration and an oxygen concentration, and a NOₓ concentration. This structure makes it possible to reduce the number of components required by the gas concentration detection device arranged in the exhaust gas pipe 71. It is also possible for the pump current detection part 512 and the oxygen concentration calculation part 513 to detect an oxygen concentration by using the pump electrode 22 and the pumping electrode 511 to be used for detecting a NOₓ concentration.

The pumping part 511, the pump current detection part 512 and the NOₓ detection part 514 are implemented including amplifiers in the sensor control unit 5. The oxygen concentration calculation part 513 and the NOₓ concentration calculation part 515 are implemented in computers arranged in the sensor control unit 5.

FIG. 1 shows, for convenience, the pumping electrode 511, the pump current detection part 512, the NOₓ detection part 514 and the potential difference detection part 521 independent from the sensor control unit 5. Actually, those parts are arranged in the inside of the sensor control unit 5. A not-shown, electrical-connection lead part of each of the electrodes 22, 23, 24, 32 and 33 is formed to the base-end side of the sensor element 10.

### (The Ammonia Concentration Calculation Part 522)

As shown in FIG. 1 and FIG. 5, the ammonia concentration calculation part 522 is configured to calculate an ammonia concentration, in the target gas G to be detected, which has been corrected based on the oxygen concentration, on the basis of the oxygen concentration detected by the oxygen concentration calculation part 513 and the potential difference ΔV calculated by the potential difference detection part 521.

FIG. 11 shows the variation in potential difference (mixed potential) ΔV due to the oxygen concentration between the ammonia electrode 32 and the second reference electrode 33 obtained by the potential difference detection part 521, detected according to the variation in ammonia concentration in the target gas G. As shown in FIG. 11, the potential difference ΔV detected (detected as a small negative value) by the potential difference detection part 521 is reduced according to the reduction of the oxygen concentration. The reason for this has already been explained by using the slope θs shown in FIG. 10.

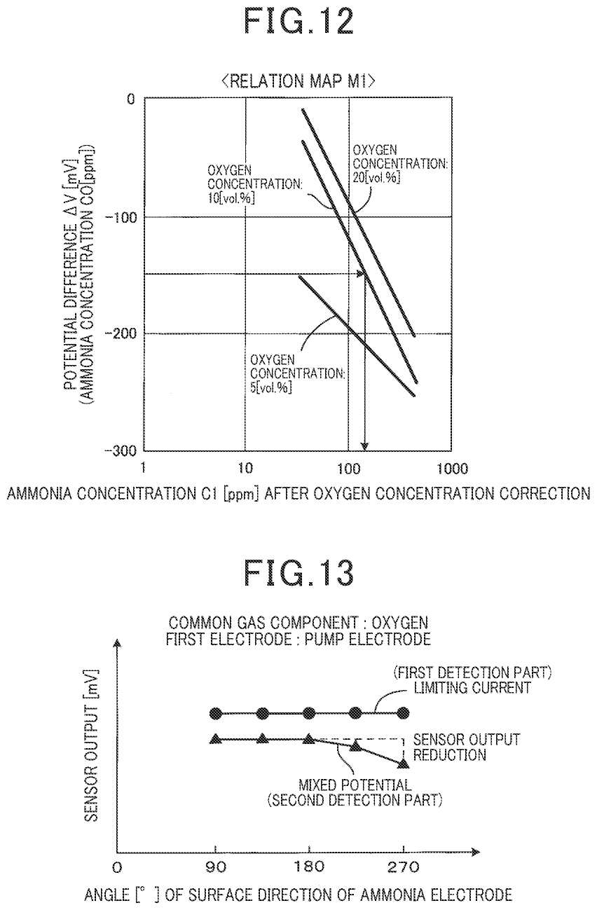

As shown in FIG. 12, the ammonia concentration calculation part 522 uses a relational map M1, using an oxygen concentration in the target gas G as a parameter. The relation map M1 shows a relationship between a potential difference ΔV detected by the potential difference detection part 521 and an ammonia concentration C1 after performing the oxygen correction which has been corrected according to an oxygen concentration. The relational map M1 is made showing a relationship between the potential difference ΔV (ammonia concentration before performing the oxygen correction) on a predetermined oxygen concentration and the ammonia concentration C1 after performing the oxygen correction. The ammonia concentration calculation part 522 collates the oxygen concentration in the target gas G and the potential difference ΔV detected by the potential difference detection part 521 with data of the relational map M1 so as to calculate the ammonia concentration C1 in the target gas G after performing oxygen correction.

More specifically, the ammonia concentration calculation part 522 collates the oxygen concentration obtained by the oxygen concentration calculation part 513 and the potential difference ΔV obtained by the potential difference detection part 521 with an oxygen concentration and a potential difference ΔV in the relational map M1. The oxygen concentration calculation part 513 reads the ammonia concentration C1 after performing oxygen correction at the potential difference ΔV in the relational map M1. The ammonia concentration calculation part 522 corrects the ammonia concentration so that the higher an oxygen concentration is, the higher an ammonia concentration C1 after performing oxygen correction. As shown in FIG. 5, the ammonia concentration C1 after performing oxygen correction becomes a corrected ammonia concentration, which has been corrected based on the oxygen concentration, output from the gas concentration detection device 1. It is acceptable to use the potential difference ΔV as the ammonia concentration CO before performing the oxygen correction in the relational map M1.

FIG. 12 shows the relational map M1 when the target gas G has the oxygen concentrations of 5 [vol. %], 10 [vol. %], and 20 [vol. %], for example. It is possible to easily correct the ammonia concentration C1 or the potential difference ΔV in accordance with the oxygen concentration. It is possible to obtain in advance the relational map M1 between the potential difference ΔV and the ammonia concentration C1 after performing oxygen correction on preparing a trial of or performing an experiment of the gas concentration detection device 1.

It is possible to prepare the relational map M1 shown in FIG. 12 at each temperature of the ammonia electrode 32. It is possible to calculate the ammonia concentration C1 after performing oxygen correction in accordance with the oxygen concentration every temperature of the ammonia electrode 32. In addition, it is possible to correct the ammonia concentration C1 after performing oxygen correction calculated by using the relational map M1 by using a temperature correction coefficient which has been determined according to a temperature of the ammonia electrode 32.

The potential difference detection part 521 and the ammonia concentration calculation part 522 are arranged in the sensor control unit (SCU) 5 electrically connected to the gas concentration detection device 1. The potential difference detection part 521 is composed of an amplifier, etc. detecting a potential difference ΔV between the ammonia electrode 32 and the second reference electrode 33. The ammonia concentration calculation part 522 is composed of a computer, etc. The sensor control unit 5 is connected to the engine control unit (ECU) 50 of the internal combustion engine 7 so as to control the operation of the internal combustion engine 7, and the reductant supply device 73, etc.

On correcting an ammonia concentration in accordance with an oxygen concentration, it is possible for the ammonia concentration calculation part 522 to correct the ammonia concentration while considering a corrected NOₓ concentration or uncorrected NOₓ concentration, detected by the NOₓ detection part 514. The NOₓ electrode 23 in the oxygen element part 2 has catalytic activity to ammonia in addition to catalytic activity to NOₓ. Accordingly, it is possible to detect, as the ammonia concentration, the uncorrected NOₓ concentration at the NOₓ electrode 23. This allows the ammonia concentration calculation part 522 to correct the ammonia concentration by using the potential difference ΔV on the basis of the oxygen concentration and the uncorrected NOₓ concentration.

### (The Heater Part 4 and the Power Supply Control Part 53)

As shown in FIG. 1 to FIG. 4, the heater part 4 is stacked on the surface of the first solid electrolyte body 21, opposite to the surface on which the second solid electrolyte body 31 is stacked. The heater part 4 heats the first solid electrolyte body 21 and the second solid electrolyte body 31. In other words, the heater part 4 is stacked at the oxygen element part 2 side, on the surface which is located opposite to the surface on which the ammonia element part 3 is stacked.

The heater part 4 is composed of the heater 41 and a heater insulator 42. The heater 41 generates heat energy when receiving electric power. The heater 41 is embedded in the heater insulator 42.

The heater insulator 42 is made of ceramic material such as alumina, etc. The reference gas duct 34, into which the reference gas A is introduced, is formed between the oxygen element part 2 and the ammonia element part 3. The reference gas duct 34 accommodates the first reference electrode 24 and the second reference electrode 33.

As shown in FIG. 1 to FIG. 4, the heater 41 is composed of a heating part 411 and a lead part 412 connected to the heating part 411. The heating part 411 is arranged at a location facing the electrodes 22, 23, 24, 32 and 33 in a stack direction, in which the solid electrolyte bodies 21 and 22, and the insulators 26, 35 and 42 are stacked. The heater 41 is connected to the power supply control part 53 which supplies electric power to the heater 41. It is possible for the power supply control part 53 to adjust a voltage to be applied to the heater 41 so as to adjust a power supply amount into the heater 41. The power supply control part 53 is composed of a drive circuit, etc. for supplying an adjusted voltage to the heater 41 based on PWM (pulse width modulation). The power supply control part 53 is arranged in the sensor control unit 5.

A distance between the ammonia element part 3 and the heater part 4 is longer than a distance between the oxygen element part 2 and the heater part 4. The heater 4 supplies heat energy to the ammonia element part 3, a temperature thereof is lower than a temperature of heat energy supplied to the oxygen element part 2. The pump electrode 22 and the NOₓ electrode 23 in the oxygen element part 2 are used at an operation temperature within a range of 600 to 900° C. The ammonia electrode 32 in the ammonia element part 3 is used at an operation temperature within a range of 400 to 600° C.

A temperature of the ammonia electrode 32 is adjusted at a temperature within an operation temperature within the range of 400 to 600° C. The power supply control part 53 is configured to adjust the operation temperature of the NOₓ electrode 23 within the range of 600 to 900° C. so as to adjust the ammonia electrode 32 to its target control temperature. This structure makes it possible for the power supply control part 53 to perform the heat control of the heater part 4 so as to heat each of the ammonia electrode 32 of the ammonia element part 3 and the NOₓ electrode 23 of the oxygen element part 2 at its necessary temperature to detect ammonia and NOₓ.

Further, the reference gas duct 34 is formed between the oxygen element part 2 and the ammonia element part 3. This structure makes it possible to use the reference gas duct 34 as an insulation layer when the heater part 4 heats the oxygen element part 2 and the ammonia element part 3. This makes it possible to reduce a temperature of the ammonia electrode 32 in the ammonia element part 3 when compared with a temperature of the NOₓ electrode 23 in the ammonia element part 3. The power supply control of the power supply control part 53 allows the oxygen element part 2 and the ammonia element part 3 to have its target temperature.

### (The Sensitivity Correction Part 54)

As shown in FIG. 7, when the sensor main body 100 having the sensor element 10 and the housing casing 61 is mounted to the attachment part 711 of the exhaust gas pipe 71, the sensor main body 100 is rotated around the attachment part 711 of the exhaust gas pipe 71 through the male screw part 611 of the housing casing 61. An arrangement location of the outer surface 311 of the sensor element 10 having the ammonia electrode 32 varies on the exhaust gas pipe 71. The sensitivity correction part 54 is configured to correct an error generated in the second detection part 52 due to an angle difference between the upstream direction J in the flow of the target gas G and a surface direction E of the outer surface 311 of the second solid electrolyte body 31.

In other words, when the sensor main body 100 is mounted to the attachment part 711 of the exhaust gas pipe 71, the sensitivity correction part 54 according to the present embodiment corrects a detection error due to a sensitivity variation on the basis of the viewpoint in which the ammonia detection sensitivity of the ammonia element part 3 as the second element part 3 having the ammonia electrode 32 varies due to a direction of the outer surface 311 of the second solid electrolyte body 31 equipped with the ammonia electrode 32 exposed toward the upstream direction J in the flow of the target gas G.

The ammonia electrode 32 of the ammonia element part 3 outputs a mixed potential (potential difference ΔV) as a sensor output when a flow of the target gas G which is in contact with the ammonia electrode 32, in other words, a necessary amount of the target gas G to be supplied to the ammonia electrode 32 is maintained. When the sensor main body 100 is mounted to the attachment part 711 of the exhaust gas pipe 71, the male screw part 611 of the housing casing 61 is engaged with the female screw part 712 of the attachment part 711. At this time, the direction of the sensor element 100 toward the upstream direction J in the flow of the target gas G is determined after the male screw part 611 is fitted to the female screw part 712 and the male screw part 611 is engaged with the female screw part 712 while the sensor main body 100 is rotated around a central axis passing through a center of the male screw part 611 and a center of the sensor element 10.

### (Relationship Between the Direction of the Sensor Element 10 and Sensor Output)

The direction of the ammonia electrode 32 toward the upstream side of the flow of exhaust gas varies according to the direction (angle) of the sensor element 10 of the sensor main body 100 attached to the attachment part 711 of the exhaust gas pipe 71. FIG. 13 shows a variation of the sensor output caused by the mixed voltage (potential difference ΔV) of the second detection part 52 using the ammonia electrode 32 and the second reference electrode 33 while changing an angle (direction) of the surface direction E of the ammonia electrode 32, arranged in the ammonia element part 3 of the sensor element 10, toward the upstream direction J in the flow of the target gas G. FIG. 13 also shows a variation amount of the sensor output due to the limiting current of the first detection part 51 using the pump electrode 22 and the first reference electrode 24.

In FIG. 13, the target gas G to be supplied to the sensor main body 100 contains ammonia at 100 ppm and oxygen of 10 vol. % in nitrogen. The target gas G to be supplied to the sensor main body 100 contains NOₓ at 100 ppm and oxygen of 10 vol. % in nitrogen when a limiting current of the first detection part 51 is detected. FIG. 13 shows a reduction amount of the sensor output of the first detection part 51 and the second detection part 52 according to a variation of the surface direction E of the ammonia electrode 32 from a reference value thereof, where the reference value of the sensor output corresponds to the output when the surface direction E of the ammonia electrode 32 has 90° toward the upstream direction J in the flow of the target gas G. That is, FIG. 13 shows the reduction amount of the sensor output of the first detection part 51 and the second detection part 52 due to a difference between the surface direction E of the ammonia electrode 32 and the reference angle of 90°. The target gas G has a constant flow rate and a constant temperature thereof during various states in which the surface direction E of the ammonia electrode 32 varies.

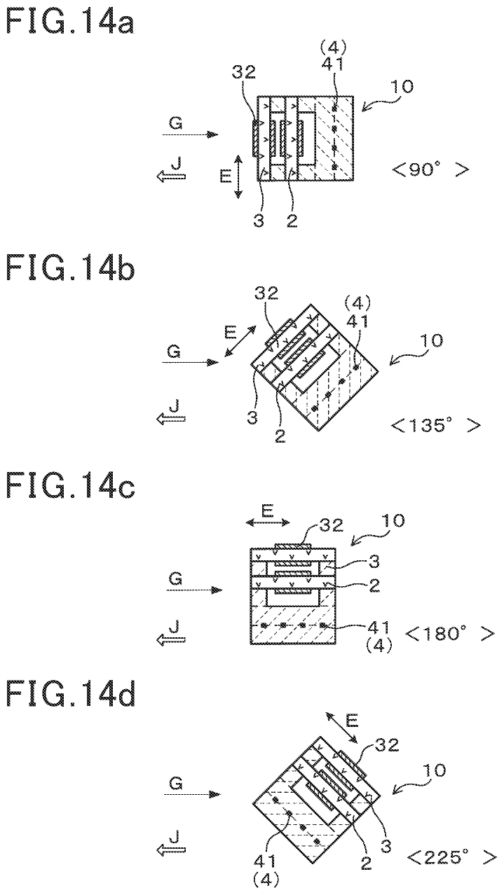

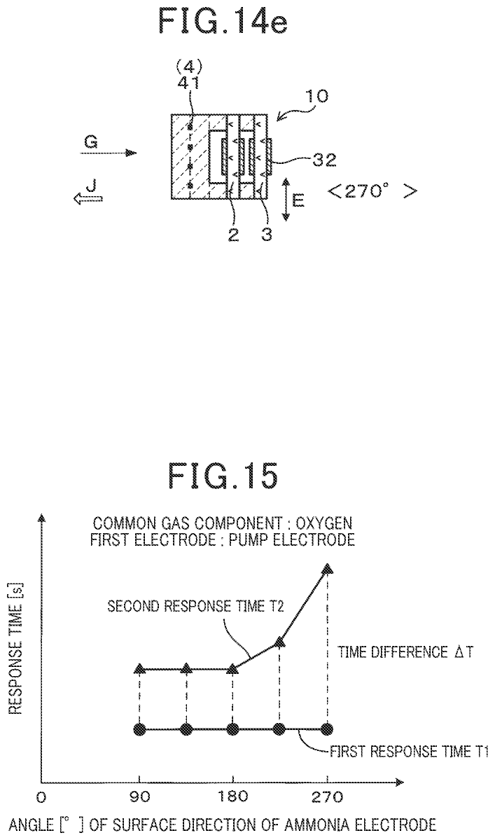

FIG. 14a shows a state in which the surface direction E of the ammonia electrode 32 of the ammonia element part 3 in the sensor element 10 has 90° toward the upstream direction J in the flow of the target gas G. This state allows exhaust gas to easily collide with the front surface of the ammonia electrode 32. That is, this state allows the exhaust gas to be easily supplied to the ammonia electrode 32. The state when the surface direction E of the ammonia electrode 32 has 45° has the same results as the state when the surface direction E of the ammonia electrode 32 is 90°.

Further, FIG. 14b shows a state in which the surface direction E of the ammonia electrode 32 of the ammonia element part 3 in the sensor element 10 has 135° toward the upstream direction J in the flow of the target gas G. FIG. 14c shows a state in which the surface direction E of the ammonia electrode 32 of the ammonia element part 3 in the sensor element 10 has 180° toward the upstream direction J in the flow of the target gas G. Those states make a condition in which the exhaust gas easily collides with the ammonia electrode 32 in a diagonal direction and a lateral direction. The state when the surface direction E of the ammonia electrode 32 has 360° is the same results as the state when the surface direction E of the ammonia electrode 32 has 180°. The state when the surface direction E of the ammonia electrode 32 has 405° is the same results as the state when the surface direction E of the ammonia electrode 32 has 135°.

FIG. 14d shows a state in which the surface direction E of the ammonia electrode 32 of the ammonia element part 3 in the sensor element 10 has 225° toward the upstream direction J in the flow of the target gas G. FIG. 14e shows a state in which the surface direction E of the ammonia electrode 32 of the ammonia element part 3 in the sensor element 10 has 270° toward the upstream direction J in the flow of the target gas G. In these states it is difficult for the exhaust gas to collide with the ammonia electrode 32. In particular, the state when the surface direction E of the ammonia electrode 32 has 270° produces the most difficulty for the exhaust gas to collide with the ammonia electrode 32. The state when the surface direction E of the ammonia electrode 32 has 315° is the same results as the state when the surface direction E of the ammonia electrode 32 has 270°.

In FIG. 13, the sensor output due to the limiting current of the first detection part 51 is substantially unchanged even if the surface direction E of the ammonia electrode 32 varies. This means that the sensor output of the first detection part 51 representing an oxygen concentration is not influenced due to a directivity of the sensor element 10. It can be understood that the sensor output of the first detection part 51 can be used to a sensitivity variation of the sensor output of the second detection part 52. The detection state, when the first detection part 51 detects a NOₓ concentration based on a limiting current generated between the NOₓ electrode 32 and the first reference electrode 24, has also the same result.

As shown in FIG. 13, the second detection part 52 provides its sensor output as the mixed potential when the surface direction E of the ammonia electrode 32 has 135° and 180° which is approximately the same as its sensor output when the surface direction E of the ammonia electrode 32 has 90° as the reference value. These cases supply an adequate amount of the target gas G to be detected to the ammonia electrode 32, and allow the second detection part 52 to correctly output a mixed potential.

On the other hand, the second detection part 52 provides its sensor output as the mixed potential when the surface direction E of the ammonia electrode 32 has 225° and 270° which is lower than the sensor output when the surface direction E of the ammonia electrode 32 has 90° as the reference value. These cases do not supply an adequate amount of the target gas G to the ammonia electrode 32, and reduce the detection sensitivity of the second detection part 52 to ammonia.

The reduction of the sensor output of the second detection part 52, which represents an ammonia concentration, is caused by the movement of the target gas G in which the target gas G flows around the sensor element 10 and arrives at the ammonia electrode 32. The reduction of the sensor output of the second detection part 52 produces a response time of the second detection part 52 when detecting a variation of the ammonia concentration. In other words, there is a strong relationship between the sensor output of the second detection part 52 and the response time of the second detection part 52. For this reason, the sensitivity correction part 54 detects a delay of the response time of the second detection part 52, and corrects a loss of sensitivity in sensor output of the second detection part 52.

### (Sensitivity Correction Coefficient K)

The sensitivity correction part 54 stores, as a sensitivity correction coefficient K, a reduction amount of the sensor output of the second detection part 52 due to the directivity of the second detection part 52. The sensitivity correction coefficient K is used to eliminate the reduction amount from the sensor output. The sensitivity correction part 54 multiples the potential difference ΔV detected by the potential difference detection part 521 with the sensitivity correction coefficient K so as to calculate the corrected potential difference ΔV. The ammonia concentration calculation part 522 calculates the corrected ammonia concentration on the basis of the corrected potential difference ΔV and the oxygen concentration. The sensitivity correction coefficient K will be explained later by using a correction map shown in FIG. 18.

(Relationship Between a Direction of the Sensor Element 10, a First Response Time T1 and a Second Response Time T2 when Oxygen is Used as a Common Gas Component)

FIG. 15 shows a second response time T2 which is required for the second detection part 52 to correctly detect a variation of an oxygen concentration based on a mixed potential (potential difference ΔV) when an angle (direction) of the surface direction E of the ammonia electrode 32 in the ammonia element part 3 in the sensor element 10 is varied the upstream direction J in the flow of the target gas G. In this case, FIG. 15 also shows a first response time T1 which is required for the pumping part 511 using the pump electrode 22 and the first reference electrode 24, and the first detection part 51 using the pump current detection part 512 and the oxygen concentration calculation part 513 to correctly detect a variation of the oxygen concentration based on a limiting current.

The first response time T1 of the first detection part 51 and the second response time T2 of the second detection part 52 are based on the reference value which is obtained when the angle of the surface direction E of the ammonia electrode 32 toward the upstream direction 3 in the flow of the target gas G is 90°. The first response time T1 and the second response time T2 represent an increased time from the reference value due to a variation of the angle of the surface direction E of the ammonia electrode 32 from 90°. The composition of the used target gas G and the angle of the surface direction of the ammonia electrode 32 toward the upstream direction J in the flow of the target gas G are the same as those shown in FIG. 13.

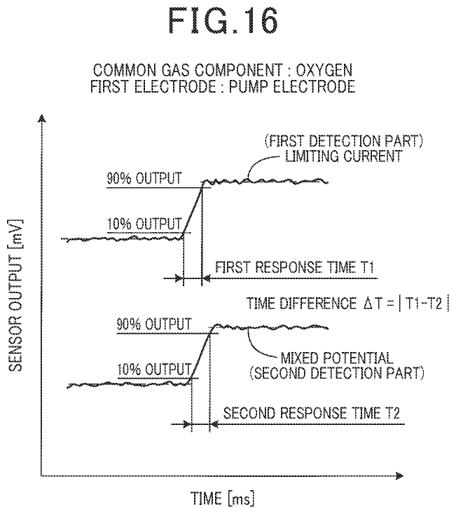

As shown in FIG. 16, the first response time T1 and the second response time T2 represent a time counted from a time when the sensor output variation changes by 10% towards the final output after a concentration change, during the variation of the oxygen concentration in the target gas G, to a time when the sensor output variation changes by 90% towards the final output after the concentration change. Each of the first response time T1 and the second response time T2 is obtained after the first detection part 51 and the second detection part 52 have detected the sensor output before variation and the sensor output after variation. It is possible to obtain each of the first response time T1 and the second response time T2 on the basis of a time difference ΔT between a time when the sensor output variation changes by 10% towards an output variation amount, which is obtained by subtracting the sensor output after variation from the sensor output before variation, and a time when the sensor output variation changes by 90%. It is possible to change the output time corresponding to the output variation amount when each of the first response time T1 and the second response time T2 is obtained.

As shown in FIG. 15, the first response time T1, which is required for the first detection part 51 detects substantially no variation of oxygen concentration even if the angle of the surface direction E of the ammonia electrode 32 toward the upstream direction J in the flow of the target gas G varies. Because the diffusion resistance part 251, through which the target gas is introduced into the inside of the gas chamber 25, is formed at the front end part of the sensor element 10, the sensor output and the first response time T1 of the first detection part 51 are not influenced by the variation of the arrangement angle of the sensor element 10 toward the upstream direction J in the flow of the target gas G. In addition, because of using a limiting current, the first detection part 51 is not influenced by the arrangement angle of the sensor element 10 toward the upstream direction J in the flow of the target gas G.

The second response time T2, when the second detection part 52 detects a variation of an ammonia concentration at the angle of the surface direction E of the ammonia electrode 32 of 135° and 180°, is substantially equal to that at the reference value of 90°. On the other hand, the second response time T2, when the second detection part 52 detects a variation of an ammonia concentration at the angle of the surface direction E of the ammonia electrode 32 of 225° and 270°, becomes longer than that at the reference value of 90°. The reason for this is the same as the case shown in FIG. 13.

The sensitivity correction part 54 corrects the ammonia concentration as the sensor output of the second detection part 52 on the basis of the time difference ΔT between the second response time T2 of the second detection part 52 and the first response time T1 of the first detection part 51. Because the first response time T1 of the first detection part 51 is not influenced by the angle of the surface direction E of the ammonia electrode 32, a delay of the second response time T2 generated in the second detection part 52 is obtained based on the first response time T1.

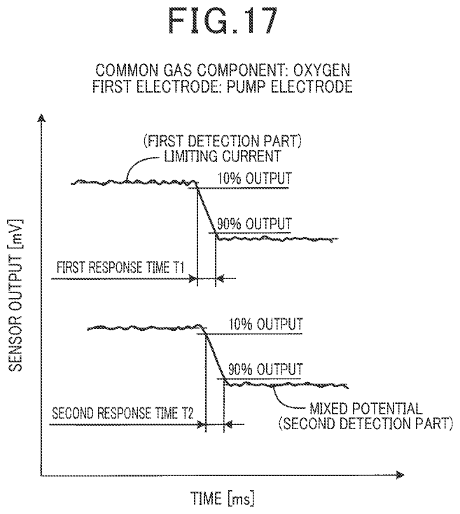

FIG. 16 shows a case in which the variation of the sensor output of each of the first detection part 51 and the second detection part 52 is increased. On the other hand, FIG. 17 shows a case in which the variation of the sensor output of each of the first detection part 51 and the second detection part 52 is reduced. Similar to the manner shown in FIG. 16, it is possible for the case shown in FIG. 17 to obtain a time difference ΔT between the first response time T1 and the second response time T2.

(Relation Graph N1 and Correction Map N2 when the Common Gas Component is Oxygen)

In a test before the initial use of the gas concentration detection device 1, it is necessary to obtain a relationship between an angle of the surface direction E of the ammonia electrode 32 toward the upstream direction J in the flow of exhaust gas and a reduction amount of the sensor output of the second detection part 52. In the test, the first detection part 51 is composed of the pumping part 511, the pump current detection part 512 and the oxygen concentration calculation part 513, and uses the pump electrode 22 and the first reference electrode 24. Oxygen is used as the common gas component to be supplied to the pump electrode 22 and the ammonia electrode 32. During the test, it is necessary to obtain a relationship between the angle of the surface direction E of the ammonia electrode 32 toward the upstream direction J in the flow of the exhaust gas, the first response time T1 and the second response time T2.

In this case, as shown in FIG. 15, the second response time T2 is detected to be larger than the first response time T1 based on the relationship in which the response speed of the second detection part 52 is slower than the response speed of the first detection part 51 detecting an oxygen concentration. It is possible to use, as an absolute value, a time difference ΔT between the first response time T1 and the second response time T2. That is, it is possible to calculate the time difference ΔT as ΔT=|T1−T2|. As shown in FIG. 18, in the test, the relation graph N1 is obtained, which represents a relationship between the sensor output and the time difference ΔT at a plurality of angles of the surface direction E of the ammonia electrode 32. In this relation graph N1, the longer the time difference ΔT is, the smaller the sensor output is.

As shown in FIG. 18, in the test, the correction map N2 is obtained as the reciprocal of the relation graph N1. In the correction map N2, the time difference ΔT is reduced according to the increase of the correction coefficient, on the basis of the maximum sensor output in the relation graph N1. This correction map N2, during the use of the gas concentration detection device 1, which expresses a reduction amount of the sensor output of the second detection part 52, which is generated when the angle of the surface direction E of the ammonia electrode 32 toward the upstream direction J in the flow of the target gas G is greater than 180°, is regulated to a magnitude of the sensor output of the regular detection part when the angle of the surface direction E is 90°.

In the test of the gas concentration detection device 1, the sensitivity correction part 54 stores the correction map N2. It is possible to obtain the sensitivity correction coefficient K as a predetermined coefficient when the time difference ΔT is referred to the correction map N2 obtained during the test before the initial use of the gas concentration detection device 1.

As shown in FIG. 18, during the use of the gas concentration detection device 1, the sensitivity correction part 54 corrects the ammonia concentration as the sensor output of the second detection part 52 by inserting the time difference ΔT, obtained on the basis of the first response time T1 and the second response time T2, into the correction map N2. This makes it possible for the sensitivity correction part 54 to correct the sensor output of the second detection part 52 representing the ammonia concentration even if the angle of the surface direction E of the ammonia electrode 32 toward the upstream direction J in the flow of the target gas G exceeds 180° when the sensor element 100 is attached to the attachment part 711 of the exhaust gas pipe 71. This makes it possible to prevent the ammonia concentration detected by the second detection part 52 from being varied by an assembled state of the sensor main body 100 in the exhaust gas pipe 71.

The detection response speed of the pump current detection part 512 detecting a pump current is higher than the response speed of the potential difference detection part 521 detecting a time difference ΔT, and is also higher than the response speed of the NOₓ detection part 514 detecting NOₓ.

### (First Output Change Time B1 and Second Output Change Time B2)

It is possible for the sensitivity correction part 54 to use various time differences ΔT, instead of using the time difference ΔT between the first response time T1 and the second response time T2. For example, as shown in FIG. 19, the sensitivity correction part 54 may use a time difference ΔT between a first output variation time B1 of the first detection part 51 and a second output variation time B2. It is possible to use, as an absolute value, the time difference ΔT between the first output variation time B1 and the second output variation time B2. That is, it is possible to use a formula ΔT=|B1−B2| as the time difference ΔT. It is possible to use each of the first output variation time B1 and the second output variation time B2, as a start time when the sensor output of each of the first detection part 51 and the second detection part 52 varies. It is possible to use, as the time difference ΔT in this case, a time difference ΔT between a start time of the first output variation of the first detection part 51 and a start time of the second output variation of the second detection part 52.

It is possible to use, as each of the first output variation time B1 and the second output variation time B2, a final time when the variation of the sensor output of each of the first detection part 51 and the second detection part 52 is completed. It is possible to use, as the time difference ΔT in this case, a time difference ΔT between a completion time of the first output variation of the first detection part 51 and a completion time of the second output variation of the second detection part 52. Further, it is possible to use, as each of the first output variation time B1 and the second output variation time B2, a time when the sensor output of each of the first detection part 51 and the second detection part 52 changes by 50% towards the final output after variation. It is possible to use, as the time difference ΔT in this case, a time difference ΔT between a time when the first output of the first detection part 51 reaches 50% and a time when the second output of the second detection part 52 reaches 50%.

### (First Response Speed U1 and Second Response Speed U2)

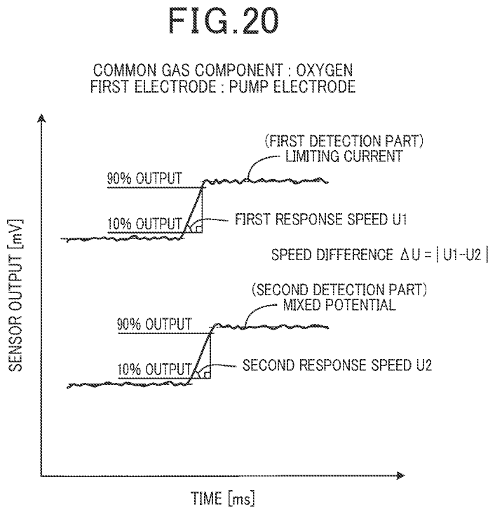

Further, it is possible for the sensitivity correction part 54 to use a speed difference ΔU, shown in FIG. 20, between the first response speed U1 of the first detection part 51 and the second response speed U2 of the second detection part 52, instead of using the time difference ΔT between the first response time T1 and the second response time T2. It is possible to use, as each of the first response speed U1 and the second response speed U2, a value obtained by dividing a variation amount, when the sensor output of each of the first detection part 51 and the second detection part 52 varies, by time. In other words, it is possible to use a slope of the relation graph N1 between the time and the sensor output as each of the first response speed U1 and the second response speed U2. It is possible to use, as an absolute value, the speed difference ΔU between the first response speed U land the second response speed U2. That is, it is possible to obtain the speed difference ΔU as ΔU=|U1−U2|. Similar to the use of each of the first response time T1 and the second response time T2, it is possible to form the relation graph N1 and the correction map N2 when each of the first response speed U1 and the second response speed U2 is used.

In the case in which the first output variation time B1 and the second output variation time B2 or the first response speed U1 and the second response speed U2 are obtained, it is acceptable for the variation of the sensor output of each of the first detection part 51 and the second detection part 52 to be reduced, in addition to be increased.

(In a Variation Formation Period in which Oxygen Concentration as the Common Gas Component Varies More than the Reference Variation Amount)

It is possible to use following various timings so as to determine the variation duration time when the concentration of the common gas varies. For example, in a case which uses the first detection part 51 for detecting an oxygen concentration, composed of the pumping part 511, the pump current detection part 512 and the oxygen concentration calculation part 513, and uses oxygen as the common gas component, it is possible to determine, as the variation duration time, a start time or a completion time of a fuel cut operation of the internal combustion engine 7 of a vehicle.

At a start time of the fuel cut operation, a fuel injection of a fuel injection device, etc., which supplies a fuel into each cylinder of the internal combustion engine 7, is stopped. At the start time, the chemical composition of exhaust gas discharged from each cylinder of the internal combustion engine 7 into the exhaust gas pipe 71 has a high oxygen concentration from a previous low oxygen concentration. It is possible for the sensitivity correction part 54 to use the correction operation based on this variation time.

At the end of the fuel cut operation, the fuel injection device, etc. restarts the fuel injection into each cylinder of the internal combustion engine 7. At this time, a chemical composition of exhaust gas discharged from each cylinder of the internal combustion engine 7 into the exhaust gas pipe 71 has a low oxygen concentration from a high oxygen concentration. It is possible for the sensitivity correction part 54 to perform the correction by using a time when the oxygen concentration in exhaust gas is changed more than the reference variation amount to a low oxygen concentration state.

For example, when, the first detection part 51 detects an oxygen concentration by using oxygen as the common reference gas component, it is possible to use, as the variation duration time, a completion time of an idling operation of the internal combustion engine 7 of a vehicle, where the first detection part 51 is composed of the pump electrode 22, the first reference electrode 24, the pumping part 511, the pump current detection part 512 and the oxygen concentration calculation part 513.

At the completion time of the idling operation, because a driver depresses the acceleration pedal of the vehicle, the fuel injection device, etc. increases an injection amount of fuel to be supplied to each cylinder of the internal combustion engine 7. At this time, a chemical composition of exhaust gas, as the target gas G to be detected, emitted from each cylinder of the internal combustion engine 7 into the exhaust gas pipe 71, is shifted to a low oxygen concentration state from a high oxygen concentration state. There is a possible variation timing at which exhaust gas has a high oxygen concentration of not less than the reference variation amount. It is possible for the sensitivity correction part 54 to perform the correction using this variation timing.

### (Timing to Obtain the Sensitivity Correction Coefficient K)

It is possible for the sensor control unit 5 to store per detection a potential difference ΔV detected by the potential difference detection part 521, a pump current (limiting current) detected by the pump current detection part 512, and a sensor current (limiting current) detected by the NOₓ detection part 514. It is possible for the sensor control unit 5 to receive a sensitivity correction signal which represents a start time or a completion time of the fuel cut operation, and a completion time of the idling operation transmitted from the engine control unit 50. Further, it is possible for the sensitivity correction part 54 to obtain a time difference ΔT between the first response time T1 and the second response time T2 on the basis of data regarding a potential difference ΔV, a pump current and a sensor current during a predetermined past period which have been stored, after the reception of the sensitivity correction signal. It is possible to obtain the sensitivity correction coefficient K on the basis of the time difference ΔT so as to correct a potential difference ΔV and an ammonia concentration.

It is also possible to obtain the first output variation time B1 and the second output variation time B2 or the first response speed U1 and the second response speed U2 by using the same manner previously described.

### (Control Method of the Gas Concentration Detection Device 1)

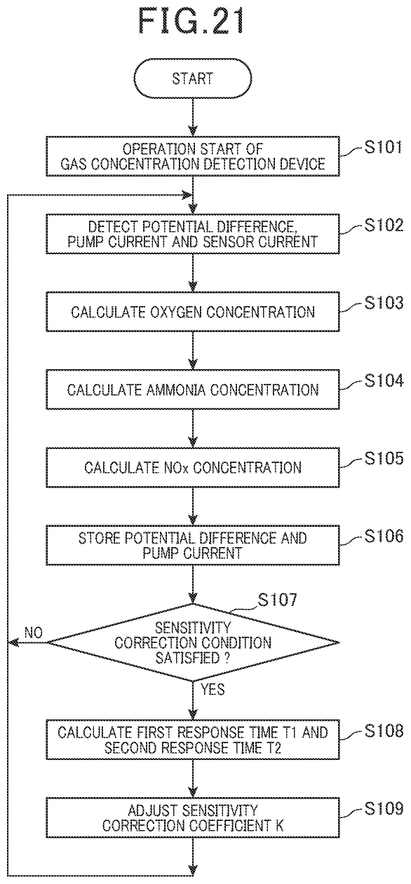

Next, a description will be given of the explanation of an example of the control method of the gas concentration detection device 1 according to the present embodiment with reference to the flow chart shown in FIG. 21.

On starting combustion of the internal combustion engine 7 when a vehicle starts, the operation of the gas concentration detection device 1 and the reductant supply device 73 also start. The power supply control part 53 in the gas concentration detection device 1 supplies electric power to the heater 41 until a temperature of the sensor element 10 is its activation temperature. After the activation of the sensor element 10, the gas concentration detection device 1 starts to perform an ammonia concentration detection, a NOₓ concentration detection and an oxygen concentration detection (step S101).

Specifically, the potential difference detection part 521 in the gas concentration detection device 1 detects a potential difference ΔV between the ammonia electrode 32 and the second reference electrode 33, and the pump current detection part 512 detects a pump current flowing between the pump electrode 22 and the first reference electrode 24. Further, the NOₓ detection part 514 detects a sensor current flowing between the NOₓ electrode 23 and first reference electrode 24 (step S102).

Further, the oxygen concentration calculation part 513 calculates an oxygen concentration in the target gas G on the basis of the pump current detected by the pump current detection part 512. Further, the ammonia concentration calculation part 522 multiplies the potential difference ΔV detected by the potential difference detection part 521 with the sensitivity correction coefficient K so as to obtain the corrected ammonia concentration of the target gas G corrected based on the oxygen concentration and the sensitivity correction coefficient K (step S104). The sensitivity correction coefficient K has a value of 1, representing no correction in an initial state before the change of the sensitivity correction coefficient K. The NOₓ concentration calculation part 515 calculates uncorrected NOₓ concentration in the target gas G on the basis of the sensor current detected by the NOₓ detection part 514. The NOₓ concentration calculation part 515 subtracts the corrected ammonia concentration from the uncorrected NOₓ concentration so as to calculate the corrected NOₓ concentration. (step S105)

The sensor control unit 5 stores the potential difference ΔV (mixed potential) detected by the potential difference detection part 521 and the pump current (limiting current) detected by the pump current detection part 512 during the predetermined period from the detection time of the potential difference ΔV and the pump current (step S106). Next, it is judged whether the sensitivity correction condition is satisfied so as to correct the sensitivity of the second detection part 52 detecting an ammonia concentration (step S107). Specifically, the sensitivity correction condition represents a time when the fuel cut operation starts, at which an oxygen concentration as the common gas component becomes not less than the predetermined reference variation amount.

When the sensitivity correction condition is satisfied, i.e. when the sensor control unit 5 receives information representing the initiation of the fuel cut operation transmitted from the engine control unit 50, the sensitivity correction part 54 obtains the first response time T1 on the basis of data regarding the pump current stored during the predetermined period, and obtains the second response time T2 on the basis of data regarding the potential difference ΔV stored during the predetermined period (step S108). Further, the sensitivity correction part 54 obtains a correction coefficient, to be used for correcting the potential difference ΔV, on the basis of the time difference ΔT between the first response time T1 and the second response time T2. The sensitivity correction part 54 corrects the correction coefficient K on the basis of the correction coefficient (step S109). The sensitivity correction coefficient K is used when the sensitivity correction part 54 inserts the time difference ΔT into the correction map N2.

Next, in step S104, when the ammonia concentration calculation part 522 calculates an ammonia concentration in the target gas G on the basis of the potential difference ΔV detected by the potential difference detection part 521, the sensitivity correction part 54 multiplies the potential difference ΔV with the sensitivity correction coefficient K so as to correct the potential difference ΔV. This makes it possible to correct an ammonia concentration error due to a sensitivity shift (reduction) of the second detection part 52 caused by an orientation of the sensor element 10 toward the upstream direction J in the flow of exhaust gas. After this, a repetition of steps S101 to S106 is performed to calculate an ammonia concentration, and the ammonia concentration is corrected by using the sensitivity correction coefficient K.

It is possible to use, as the sensitivity correction coefficient K, a mean value of a plurality of values obtained when the sensitivity correction condition in step S107 is satisfied. On stopping the combustion of the internal combustion engine 7, the gas concentration detection device 1 completes the detection of an ammonia concentration, a NOₓ concentration, and an oxygen concentration.

### (Action and Effects)

The gas concentration detection device 1 according to the present embodiment has the first detection part 51 and the second detection part 52. The first detection part 51 detects an oxygen concentration on the basis of a limiting current (pump current). The second detection part 52 detects an ammonia concentration on the basis of a potential difference (mixed potential) ΔV. The gas concentration detection device 1 corrects the ammonia concentration detected by the second detection part 52 by using a time difference ΔT between the first response time T1 of the first detection part 51 and the second response time T2 of the second detection part 52 when the first detection part 51 and the second detection part 52 detects oxygen as the common gas component.

The pumping part 511, the pump current detection part 512 and the oxygen concentration calculation part 513 forming the first detection part 51 use a limiting current detected when a flow rate of the target gas G is regulated (limited) and a direct current voltage is supplied between the pump electrode 22 and the first reference electrode 24. Accordingly, the oxygen concentration calculation part 513 detects an oxygen concentration without any influence of the catalyst performance of the pump electrode 22. The oxygen concentration calculation part 513 may detect an oxygen concentration with substantially no errors.

On the other hand, the second detection part 52 composed of the potential difference detection part 521 and the ammonia concentration calculation part 522 uses a potential difference ΔV detected when the ammonia electrode 32 is in contact with the target gas G. Accordingly, the detection of the potential difference ΔV is drastically influenced by the catalyst performance of the ammonia electrode 32. The ammonia concentration calculation part 522 easily produces an ammonia concentration detection error.

The sensitivity correction part 54 uses the first response time T1, to be used when the first detection part 51 detects an oxygen concentration, i.e. detects a concentration of the common gas component, as the reference time so as to correct the detection sensitivity of the second detection part 52. The sensitivity correction part 54 detects a time difference of the second response time T2 from the first response time T1, where the second response time T2 is detected when the second detection part 52 detects an oxygen concentration as the common gas component. The sensitivity correction part 54 determines a correction amount of the ammonia concentration detected by the second detection part 52 on the basis of the detected time difference.

More specifically, the sensitivity correction part 54 obtains the first response time T1 and the second response time T2 when a concentration of oxygen as the common gas component, which is in contact with the pump electrode 22 and the ammonia electrode 32, is varied not less than the predetermined reference variation amount. The sensitivity of the potential difference detection part 521 to ammonia is influenced by the catalyst performance of the ammonia electrode 32. There is a relationship between a time difference ΔT between the first response time T1 and the second response time T2, and the detection sensitivity of the potential difference detection part 521 when detecting ammonia.

The sensitivity correction part 54 in the gas concentration detection device 1 according to the present embodiment uses a relationship between the time difference ΔT and the sensitivity of the potential difference detection part 521 to ammonia. The sensitivity correction part 54 uses the time difference ΔT so as to correct the ammonia concentration detected by the ammonia concentration calculation part 522 because the time difference ΔT represents the detection sensitivity of the potential difference detection part 521 to ammonia.

The sensitivity of the potential difference detection part 521 to ammonia varies due to the influence of the angle of the surface direction E of the ammonia electrode 32 toward the upstream direction 3 in the flow of exhaust gas. Because of this directivity, the sensitivity correction part 54 corrects the ammonia concentration detected by the ammonia concentration calculation part 522. It is accordingly possible for the gas concentration detection device 1 to reduce a detection error of the ammonia concentration as the output thereof.

Accordingly, it is possible for the gas concentration detection device 1 according to the present embodiment to prevent an error of the ammonia output concentration obtained based on the potential difference ΔV from being caused when detecting the oxygen concentration and ammonia concentration.

It is possible to use nitrogen dioxide (NO₂), instead of using ammonia to be detected as the second gas component by the second detection part 52. This case uses an electrode having catalytic activity to nitrogen dioxide as the mixed potential electrode in the second element part 3. In this case, the mixed potential electrode detects a mixed potential generated when the electrochemical reduction reaction of oxygen contained in the target gas G is balanced with the electrochemical oxidation reaction of nitrogen dioxide contained in the target gas G. This case may obtain the same actions and effects as the case which detects an ammonia concentration previously explained.

### Second Embodiment

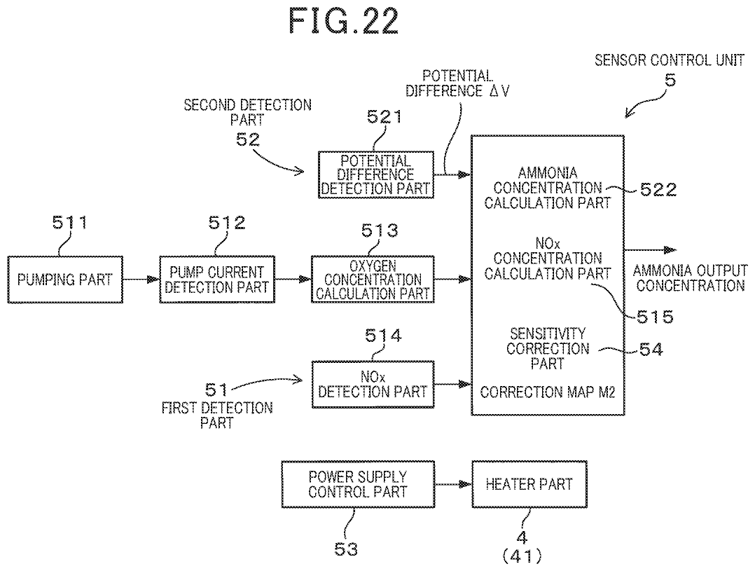

As shown in FIG. 22, the gas concentration detection device 1 according to the present embodiment shows a structure in which the first detection part 51 is composed of the NOₓ detection part 514 and the NOₓ concentration calculation part 515, and the first detection part detects a NOₓ concentration as the first gas component concentration. In the gas concentration detection device 1 according to the present embodiment, the first detection part 51 detects a NOₓ concentration instead of correcting the second gas component concentration detected by the second detection part 52. The sensor element 10 according to the present embodiment has a structure which is the same as that according to the first embodiment.

The sensitivity correction part 54 according to the present embodiment uses ammonia as the common gas component which is sensitive to the NOₓ detection part 514 and the potential difference detection part 521. For example, ammonia is oxidized at a high temperature of not less than 600° C. to generate NOₓ. The first solid electrolyte body 21, the pump electrode 22 and the diffusion resistance part 251 which have been heated at a temperature of not less than 600° C. oxides ammonia to NOₓ, and NOₓ migrates and reaches the NOₓ electrode 23. It is accordingly possible to use ammonia as the common gas component, to which the NOₓ detection part 514 and the potential difference detection part 521 have the detection sensitivity.

When ammonia is used as the common gas component, the first response time T1 is a time required for the first detection part 51 to detect an ammonia concentration by using the NOₓ electrode 23, the first reference electrode 24, the NOₓ detection part 514 and the NOₓ concentration calculation part 515. The first detection part 51 detects NOₓ generated from the oxidation of ammonia. The second response time T2 is a time which is necessary for the second detection part 52 to detect a variation of an ammonia concentration on the basis of a mixed potential (potential difference ΔV).

(Relation Graph N1 and Correction Map N2 when Ammonia is the Common Gas Component)

In a test before its initial use, the gas concentration detection device 1 according to the present embodiment obtains a relationship between a reduction amount of the sensor output of the second detection part 52 and the angle of the surface direction E of the ammonia electrode 32 toward the upstream direction J in the flow of exhaust gas in the exhaust gas pipe 71. In the test, the gas concentration detection device 1 obtains a relationship between a time difference ΔT between the first response time T1 and the second response time T2, and the angle of the surface direction E of the ammonia electrode 32 toward the upstream direction J in the flow of exhaust gas.

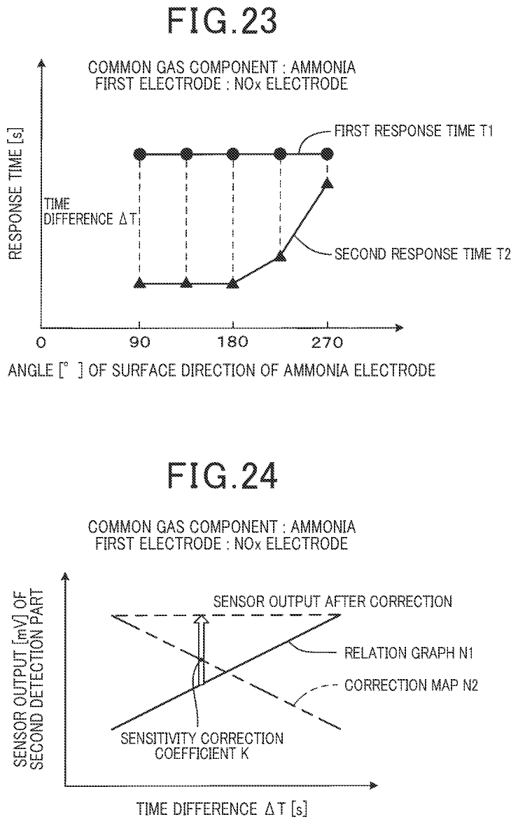

As shown in FIG. 23, in this case in which the first detection part 51 detecting NOₓ produced by oxidation of ammonia has a response speed which is slower than a response speed of the second detection part 52, the first response time T1 becomes larger than the second response time T2. It is possible to use, as an absolute value, the time difference ΔT between the first response time T1 and the second response time T2. As shown in FIG. 24, with the surface direction E of the ammonia electrode 32 during the test having a plurality of angles, the relation graph N1 between the sensor output and the time difference ΔT is obtained. This relation graph N1 represents a relationship where the longer the time difference ΔT is, the larger the sensor output is.

As shown in FIG. 24, in the test, the correction map N2 is obtained as the reciprocal of the relation graph N1. In the correction map N2, the time difference ΔT is reduced according to the increase of the correction coefficient, on the basis of the maximum sensor output in the relation graph N1. This correction map N2, during the use of the gas concentration detection device 1, which expresses a reduction amount of the sensor output of the second detection part 52, which is generated when the angle of the surface direction E of the ammonia electrode 32 toward the upstream direction J in the flow of exhaust gas exceeds 180°, is regulated to a magnitude of the sensor output of the regular detection part. In the test of the gas concentration detection device 1, the sensitivity correction part 54 stores the correction map N2.

(Variation Formation Period in which a Concentration of Ammonia as the Common Gas Component is Changed not Less than the Reference Variation Amount)

When the first detection part 51 composed of the NOₓ detection part 514 and the NOₓ concentration calculation part 515 uses ammonia as the common gas component so as to detect an ammonia concentration, it is possible for the first detection part 51 to determine the variation formation period in which a concentration of ammonia as the common gas component is varied by not less than the reference variation amount during a fuel cut operation or an idling operation of the internal combustion engine 7 of a vehicle.

Ammonia as the reductant K in the catalyst 72, supplied from the reductant supply device 73 to the exhaust gas pipe 71 of the internal combustion engine 7, is discharged into the exhaust gas pipe 71 when ammonia is not adhered on the catalyst 72. At this time, there is a possible case in which the concentration of ammonia as the common gas component detected by the second detection part 52 is increased by not less than the reference variation amount. Further, when an ammonia amount provided from the catalyst 72 into the exhaust gas pipe 71 is reduced, there is a possible case in which the concentration of ammonia as the common gas component detected by the second detection part 52 is reduced by not less than the reference variation amount. It is possible for the sensitivity correction part 54 to use this variation time so as to perform its correction operation.

If the reductant supply device 73 injects a urea water containing an excess amount of ammonia to be adhered on the catalyst 72, it is possible to use, as the common gas component, the excess ammonia discharged from the catalyst 72 into the target gas G as the common gas component. This case makes it possible for an ammonia concentration variation of not less than the reference variation amount in the target gas G to occur.

The gas concentration detection device 1 according to the present embodiment performs the same control operation as the gas concentration detection device 1 according to the first embodiment. It is possible for the present embodiment to use the variation formation period, which satisfies the sensitivity correction condition in step S107 shown in FIG. 21, in the fuel cut operation or the idling operation.

When the ammonia electrode 32 detects NOₓ in the target gas G, it is acceptable for the sensitivity correction part 54 to use NOₓ as the common gas component.

Other components, action and effects, etc. of the gas concentration detection device 1 according to the present embodiment are the same as those of the gas concentration detection device 1 according to the first embodiment. The same reference numbers and characters in the present embodiment and the first embodiment represent the same components.

### Third Embodiment

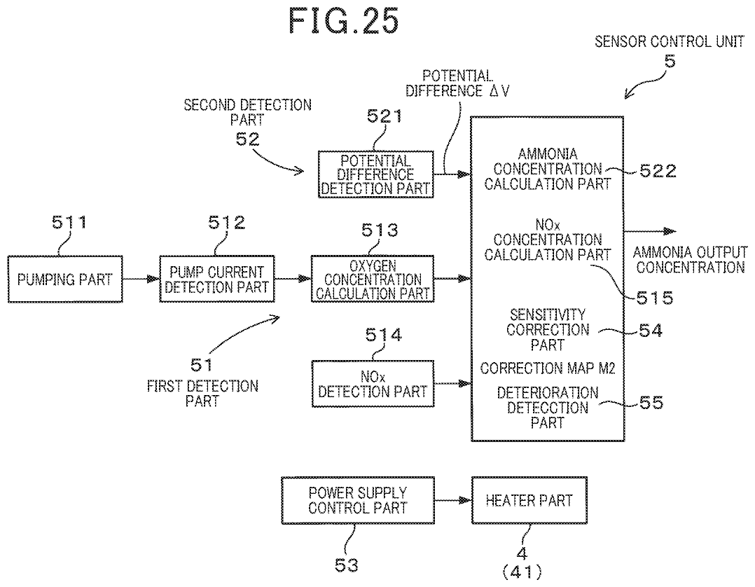

As shown in FIG. 25, the gas concentration detection device 1 according to the present embodiment has a deterioration detection part 55 in addition to the sensitivity correction part 54, etc. The deterioration detection part 55 detects occurrence of deterioration of the ammonia element part 3 as the second element part 3. The deterioration detection part 55 detects a time difference ΔTc between an initial time difference ΔTa and a use time difference ΔTb when a chemical composition of exhaust gas as the target gas G emitted from the internal combustion engine 7 to the exhaust gas pipe 71 is slightly varied. The initial time difference ΔTa is obtained at the initial use of the gas concentration detection device 1. The use time difference ΔTb is obtained after the elapse of a predetermined use time counted from the initial use of the gas concentration detection device 1.

The initial time difference ΔTa is obtained as a time difference ΔT, at the initial use of the gas concentration detection device 1, between the first response time T1 of the first detection part 51 and the second response time T2 of the second detection part 52. The use time difference Δb is obtained as a time difference ΔT, after the elapse of the predetermined use time of the gas concentration detection device 1, between the first response time T1 of the first detection part 51 and the second response time T2 of the second detection part 52.

It is possible for the deterioration detection part 55 to judge that the ammonia element part 2 has been deteriorated when the time difference ΔTc between the initial time difference ΔTa and the use time difference ΔTb is more than a predetermined time difference Δ0. Further, it is possible for the deterioration detection part 55 to judge that no deterioration occurs in the ammonia element 3 or the deteriorated state of the ammonia element 3 is acceptable when the time difference ΔTc between the initial time difference ΔTa and the use time difference ΔTb is within the predetermined time difference Δ0. It is possible to obtain the time difference ΔTc as an absolute value represented by a formula ΔTc=|ΔTa−ΔTb|.

It is possible to use, as the predetermined time to be used for determining the use time difference ΔTb, a time when a vehicle equipped with the gas concentration detection device 1 has driven for a predetermined distance. It is possible for the deterioration detection part 55 to detect a deterioration state of the ammonia element part 3 on the basis of the time difference ΔTc between the initial time difference ΔTa and the use time difference ΔTb, a ratio of the use time difference ΔTb to the initial time difference ΔTa, etc.

Similar to the case of the first embodiment, it is possible for the deterioration detection part 55 to use the first output variation time B1 and the second output variation time B2 instead of using the first response time T1 and the second response time T2. In this case, it is possible for the deterioration detection part 55 to judge the presence of deterioration or a degree of deterioration on the basis of the time difference ΔTc. The time difference ΔTc is a time difference between the initial time difference ΔTa and the use time difference ΔTb, where the initial time difference ΔTa represents a time difference between the first output variation time B1 detected by the first detection part 51 and the second output variation time B2 detected by the second detection part 52 at the initial use of the gas concentration detection device 1, and the use time difference ΔTb represents a time difference between the first output variation time B1 detected by the first detection part 51 and the second output variation time B2 detected by the second detection part 52 after the elapse of the predetermined time counted from the initial use time of the gas concentration detection device 1.

Similar to the first embodiment, it is possible for the deterioration detection part 55 to use the first response speed U1 and the second response speed U2 instead of using the first response time T1 and the second response time T2. In this case, it is possible for the deterioration detection part 55 to judge the presence of deterioration or a degree of deterioration on the basis of a speed difference ΔUc between an initial speed difference ΔUa and a use speed difference ΔUb, where the initial speed difference ΔUa represents a speed difference between the first response speed U1 of the first detection part 51 and the second response speed U2 of the second detection part 52 obtained at the initial use of the gas concentration detection device 1, and the use speed difference ΔUb represents a speed difference between the first response speed U1 of the first detection part 51 and the second response speed U2 of the second detection part 52 after the elapse of the predetermined time counted from the initial use time of the gas concentration detection device 1.

When oxygen is used as the common gas component, similar to the first embodiment, it is possible to form the first detection part 51 by using the pumping part 511, the pump current detection part 512 and the oxygen concentration calculation part 513. When ammonia is used as the common gas component, similar to the second embodiment, it is possible to form the first detection part 51 by using the NOₓ detection part 514 and the NOₓ concentration calculation part 515.

### (Control Method of the Gas Concentration Detection Device 1)

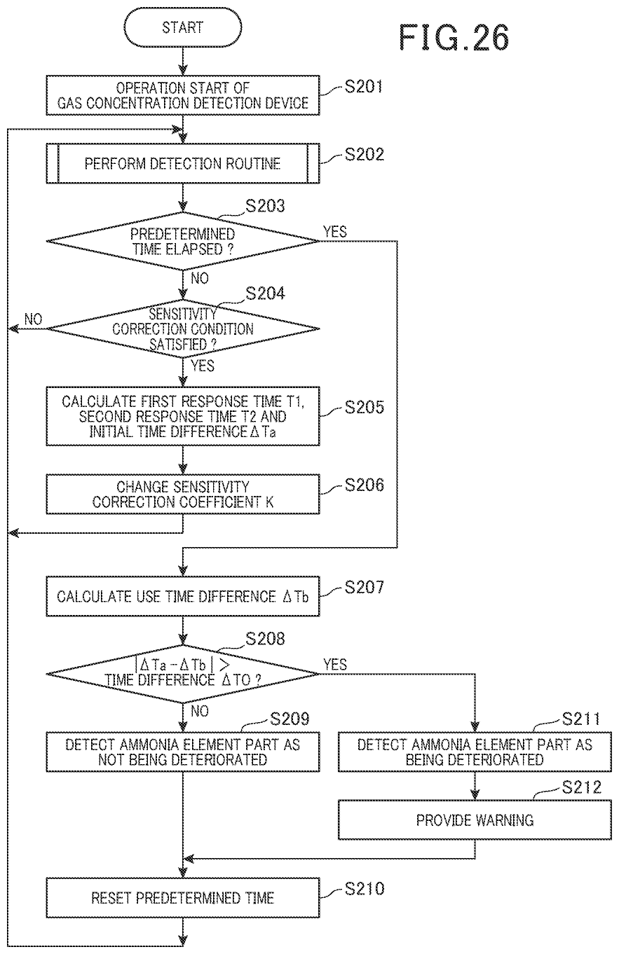

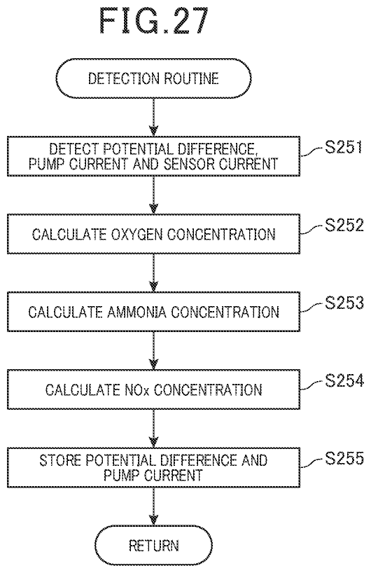

Next, a description will be given of one example of the control method of the gas concentration detection device 1 according to the present embodiment with reference to the flow chart shown in FIG. 26 and FIG. 27.

Similar to step S101 shown in FIG. 21 according to the first embodiment, after the activation of the sensor element 10, the gas concentration detection device 1 starts to perform an ammonia concentration detection, a NOₓ concentration detection and an oxygen concentration detection (step S201 shown in FIG. 26). On starting combustion of the internal combustion engine 7 when a vehicle starts, the operation of the gas concentration detection device 1 and the reductant supply device 73 also start. The power supply control part 53 in the gas concentration detection device 1 supplies electric power to the heater 41 until a temperature of the sensor element 10 reaches its activation temperature. After the activation of the sensor element 10, the gas concentration detection device 1 starts the detection of an ammonia concentration, a NOₓ concentration and an oxygen concentration (step S101). Next, step S251 to step S255 are performed as the detection routine, similar to step S101 to step S106 shown in FIG. 21 according to the first embodiment.

Next, after the operation of the gas concentration detection device 1, it is detected whether the predetermined time period has been elapsed so as to perform the deterioration detection (step S203). Before the elapse of the predetermined time period, it is detected whether the sensitivity correction condition is satisfied so as for the second detection part 52 to correct the sensitivity, where the second detection part 52 detects an ammonia concentration (step S204). Similar to step S108 and step S109 shown in FIG. 21 according to the first embodiment, when the sensitivity correction condition is satisfied, step S205 and step S206 shown in FIG. 26 are performed. In step S205 and step S206, the initial time difference ΔTa represents a time difference between the first response time T1 and the second response time T2 detected by the sensitivity correction part 54.

On the other hand, in step S203, when detecting the elapse of the predetermined time period, the sensitivity correction part 54 obtains, as the time difference ΔTb, a time difference ΔT between the first response time T1 of the first detection part 51 and the second response time T2 of the second detection part 52 (step S207). Next, the deterioration detection part 55 detects whether the time difference ΔTc between the initial time difference ΔTa and the use time difference ΔTb exceeds the predetermined time difference ΔT0 (step S208). The time difference ΔTc is obtained by using a formula |ΔTa−ΔTb|.

In step S208, when the time difference ΔTc is within the predetermined time difference ΔT0, the deterioration detection part 55 detects that no deterioration occurs in the ammonia element part 3 (step S209). The deterioration detection part 55 resets the predetermined time to be used for determining a deterioration determination period (step S210) so as to perform a repetition of the detection routine of step S202.

In step S208, when the time difference ΔTc exceeds the predetermined time difference ΔT0, the deterioration detection part 55 provides information regarding the occurrence of deterioration of the ammonia element part 3 (step S211). It is possible for the deterioration detection part 55 to use a lamp display so as to provide warning of the occurrence of deterioration of the ammonia element part 3 (step S212). In this case, the deterioration detection part 55 resets the predetermined time to be used for determining a deterioration determination period (step S210) so as to perform a repetition of the detection routine of step S202.

### (Action and Effects)

The ammonia electrode 32 in the ammonia element part 3 detects a mixed potential, and deterioration of the catalyst performance easily influences its detection accuracy. The deterioration of the ammonia electrode 32 progresses due to heat energy, and toxic substances, etc. contained in exhaust gas as the target gas G. The ammonia concentration detection by the second detection part 52 in the ammonia element part 3 easily produces a detection error due to the catalyst performance of the ammonia electrode 32.

In the gas concentration detection device 1 according to the present embodiment, the sensitivity correction part 54 corrects the sensitivity of the sensitivity correction part 54, and the deterioration detection part 55 detects occurrence of deterioration of the ammonia element part 3. When the deterioration reducing the ammonia concentration detection accuracy occurs in the ammonia element part 3, the gas concentration detection device 1 generates a warning to the driver of a vehicle so as to perform a maintenance operation of the gas concentration detection device 1.

Other components, action and effects, etc. of the gas concentration detection device 1 according to the present embodiment are the same as those of the gas concentration detection device 1 according to the first and second embodiments. The same reference numbers and characters in the present embodiment and the first embodiment represent the same components.

### Fourth Embodiment

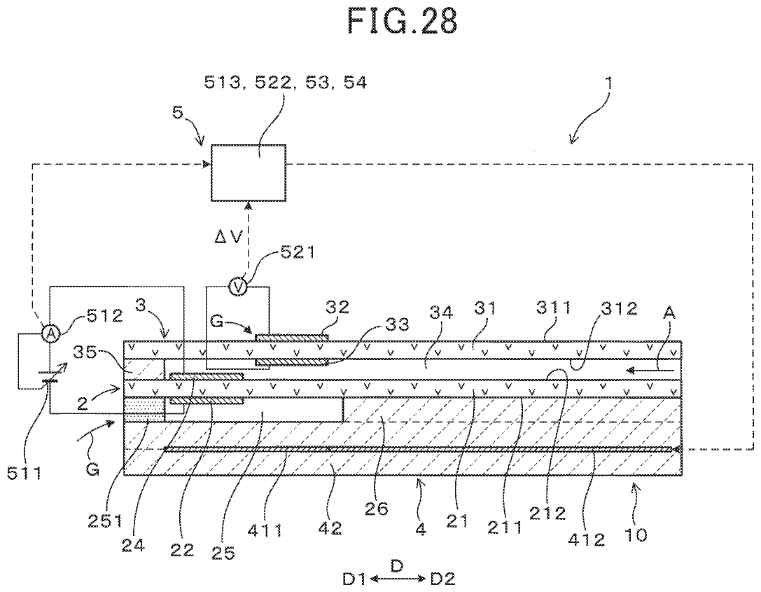

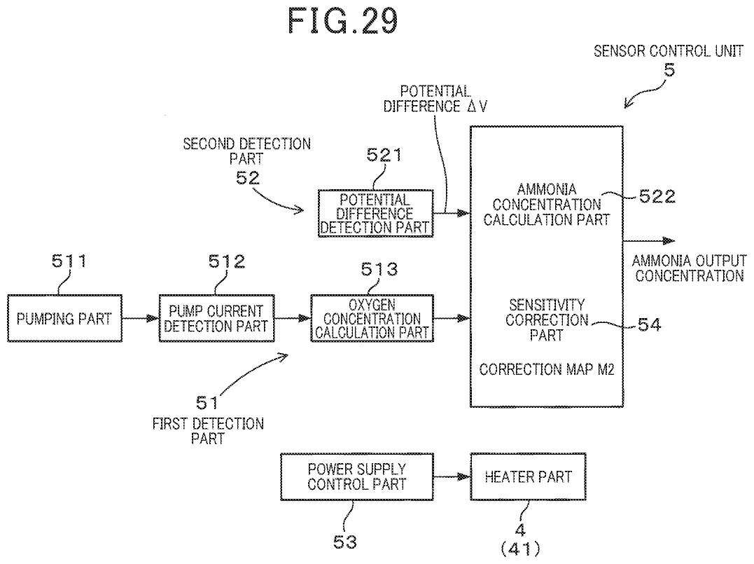

The present embodiment shows the sensor element 10 for detecting an oxygen concentration and an ammonia concentration. On the other hand, the sensor element 10 does not have a NOₓ detection function. As shown in FIG. 28 and FIG. 29, the pump electrode 22 and the first reference electrode 24 are formed on the first solid electrolyte body 21. No NOₓ electrode 23 is formed on the first solid electrolyte body 21. The oxygen element part 2 is composed of the first solid electrolyte body 21, the pump electrode 22, the first reference electrode 24, the gas chamber 25 and the diffusion resistance part 251. The ammonia element part 3 according to the present embodiment has the same structure as that according to the first embodiment.

The first detection part 51 according to the present embodiment is composed of the pumping part 511, the pump current detection part 512 and the oxygen concentration calculation part 513 so as to detect an oxygen concentration in the target gas G to be detected. The sensitivity correction part 54 according to the present embodiment has the same structure of that according to the first embodiment. The gas concentration detection device 1 according to the present embodiment has the same structure as that according to the first embodiment excepting for the NOₓ detection function.

Other components, action and effects, etc. of the gas concentration detection device 1 according to the present embodiment are the same as those of the gas concentration detection device 1 according to the first, second and third embodiments. The same reference numbers and characters in the present embodiment and the first, second and third embodiments represent the same components.

It is possible to avoid the reference gas duct 34 from the sensor element 10 in the gas concentration detection device 1 according to the first to fourth embodiments.

Further, it is possible to eliminate the second reference electrode 33 in the ammonia element part 3 from the reference gas duct 34. In this case, it is possible to arrange the ammonia electrode 32 and the second reference electrode 33 on the outer surface 311 of the second solid electrolyte body 31. The outer surface 311 of the second solid electrolyte body 31 forms the outer surface of the sensor element 10. This case makes it possible to detect an ammonia concentration in the target gas G on the basis of a difference in catalyst activation between the ammonia electrode 32 and the second reference electrode 33.

As previously described in detail, the present disclosure provides the gas concentration detection device for detecting a gas concentration of at least two kinds of gas components, and preventing a detection error of a concentration of a secondary gas component detected based on a potential difference from occurring. That is, the inventors of the present disclosure have found that a detection sensitivity of the ammonia detection part in the sensor element of the sensor main body varies with arrangement direction in the exhaust gas pipe. Further, the inventors of the present disclosure have found that an ammonia concentration detected by a gas concentration device equipped with the sensor main body may contain an error caused by a detection sensitivity reduction due to its arrangement direction.

The multi-component gas sensor previously described does not consider any variation of the detection sensitivity of the ammonia detection part due to its arrangement direction. On the other hand, a general gas sensor is configured to have a structure in which a sensor element with a cover having communication holes is mounted on a housing casing. A target gas to be detected flows into the inside of the housing casing through the communication holes. It is possible to adjust a flow rate of the target gas collided with the sensor element in the housing casing. However, the use of such a cover provides a less reduction of the detection sensitivity of the ammonia detection part. This is difficult to adequately correct the reduction of the detection sensitivity of the ammonia detection part.

In the gas concentration detection device in accordance with one aspect of the present disclosure, the detection sensitivity of the second detection part detecting the second gas component on the basis of a potential difference generated during its operation is corrected. This makes it possible to prevent a detection error of a detected second gas component concentration from occurring. In more specifically, the gas concentration detection device has the first detection part and the second detection part. The first detection part detects a first gas component concentration on the basis of a limiting current. The second detection part detects a second gas component concentration on the basis of a potential difference. When the first detection part and the second detection part detect a variation in concentration of a common gas component, the gas concentration detection device corrects the second gas component concentration detected by the second detection part on the basis of the output variation time difference between the first detection part and the second detection part, the response time difference between the first detection part and the second detection part or the response speed difference between the first detection part and the second detection part.

That is, the output variation time difference indicates a difference between the first output variation time B1 detected by the first detection part and the second output variation time B2 detected by the second detection part, the response time difference indicates a difference between the first response time T1 of the first detection part and the second response time T2 of the second detection part, and the response speed difference indicates a difference between the first response speed U1 of the first detection part and the second response speed U2 of the second detection part.

The first detection part uses a limiting current detected when a flow amount of the target gas is limited and a direct current voltage is applied between the pair of first electrodes. Catalyst performance of the pair of first electrodes does not substantially affect the detection of the first gas component concentration detected by the first detection part. The first detection part detects the first gas component concentration without an error caused by the catalyst performance of the pair of first electrodes.

On the other hand, the second detection part uses a potential difference detected when the target gas is in contact with the pair of second electrodes. Catalyst performance of the pair of second electrodes drastically affects the detection of the second gas component concentration detected by the second detection part. Accordingly, the second detection part often produces a detection error of the second gas component concentration due to the catalyst performance of the pair of second electrodes.

The sensitivity correction part performs its correction operation on the basis of the first output variation time obtained when the first detection part detects the common gas component and the first response time or the first response speed. The sensitivity correction part detects a difference between the first output variation time and the second output variation time, a difference between the first response time and the second response time, or a difference between the first response speed and the second response speed when the second detection part detects the common gas component. The sensitivity correction part corrects the second gas component concentration detected by the second detection part on the basis of the detected differences previously described.

In more specifically, the sensitivity correction part obtains the first output variation time and the second output variation time, the first response time and the second response time or the first response speed and the second response speed when a concentration of the second gas component, which is in contact with the pair of first electrodes in the first element part and the pair of second electrodes in the second element part, is not less than the reference variation amount. The catalyst performance of the pair of second electrodes affects the second output variation time, the second response time, the second response speed, and the sensitivity of detecting the second gas component concentration when the second detection part detects the second gas component concentration. There is a relationship between the sensitivity when the second detection part detects the second gas component concentration, and a time difference between first output variation time and the second output variation time, a time difference between first response time and the second response time and a speed difference between the first response speed and the second response speed.

The sensitivity correction part in the gas concentration detection device in accordance with one aspect of the present disclosure uses the relationship between the sensitivity of the second detection part and the time differences or the speed difference. The sensitivity correction part performs the correction operation of the detected second gas component concentration on the basis of the time differences or the speed difference by influencing the detection sensitivity of the second detection part. This makes it possible for the sensitivity correction part to correct the second gas component concentration and to prevent an error of the second gas component concentration provided by the gas concentration detection device from occurring even if the reduction of the detection sensitivity of the second detection part occurs when detecting the second gas component affected from an assembled condition of the gas concentration detection device

Accordingly, the gas concentration detection device according to one aspect of the present disclosure makes it possible to prevent an error of the second gas component concentration detected based on a potential difference from occurring when detecting at least two types of gas components.

It is possible for the sensitivity correction part to obtain a sensitivity correction coefficient, so as to correct the potential difference and the second gas component concentration obtained by the second detection part, on the basis of the time difference, etc. between the first response time and the second response time, etc. The sensitivity correction part multiplies the potential difference or the second gas component concentration by a sensitivity correction coefficient, and corrects the second gas component concentration based on this multiplied result. In the present disclosure, the correction of the potential difference is equivalent to the correction of the second gas component concentration. It is also possible for the sensitivity correction part to use the sensitivity correction coefficient in order to obtain a time difference between the first output variation time and the second output variation time or a speed difference between the first response speed and the second response speed.

The sensitivity correction part detects a time difference between the first response time and the second response time, and other time differences, one or multiple times during an initial use state of the gas concentration detection device. It is possible for the sensitivity correction part to obtain the sensitivity correction coefficient based on the detected time differences. This makes it possible for the sensitivity correction part to sequentially correct the second gas component concentration based on multiple results of the potential difference or the second gas component concentration, sequentially detected by the second detection part, with the obtained sensitivity correction coefficient. It is also possible to use the sensitivity correction coefficient so as to obtain the time difference between the first output variation time and the second output variation time and to obtain the speed difference between the first response time and the second response time.

While each of specific embodiments of the present disclosure has been described in detail, it will be appreciated by those skilled in the art that various modifications and alternatives to those details could be developed in light of the overall teachings of the disclosure. Accordingly, the particular arrangements disclosed are meant to be illustrative only and not limited to the scope of the present disclosure which is to be given the full breadth of the following claims and all equivalents thereof. Signs in parentheses, which indicate structural components of the present disclosure, indicate a relationship between signs used in the description of the embodiments and signs in drawings. These signs do not limit the scope of the present disclosure.

What is claimed is:

1. A gas concentration detection device comprising:
a first element part, a first detection part, a second element part, a second detection part and a sensitivity correction part, wherein
the first element part comprises a first solid electrolyte body having ionic conductivity, a pair of first electrodes arranged on the first solid electrolyte body, and, a diffusion resistance part and a gas chamber, the gas chamber accommodates one of the pair of first electrodes, the gas chamber being configurated to receive, via the diffusion resistance part, a target gas to be detected,
the first detection part is configured to detect a first gas concentration contained in the target gas on the basis of a direct current flowing between the pair of first electrodes when a flow amount of the target gas into the gas chamber is adjusted by the diffusion resistance part and a direct current voltage is applied to the pair of first electrodes,
the second element part comprises a second solid electrolyte body having ionic conductivity and a pair of second electrodes, the second solid electrolyte body is laminated on the first electrolyte body through an insulator, and the pair of second electrodes are arranged on the second solid electrolyte body,
the second detection part is configured to detect a second gas component concentration contained in the target gas on the basis of a potential difference generated between the pair of second electrodes, when at least one of the pair of second electrodes being arranged on an outer surface of the second electrolyte body and exposed to the target gas, in response to the first detection part and the second detection part detecting a variation in concentration of a common gas component, contained in the target gas, of more than a reference variation amount, contained in the target gas,
the sensitivity correction part is configured to correct the second gas component concentration detected by the second detection part on the basis of an output variation time difference and either of a response time difference or a response speed difference, the output variation time difference indicates a difference between a first output variation time detected by the first detection part and a second output variation time detected by the second detection part, the response time difference indicates a difference between a first response time of the first detection part and a second response time of the second detection part, and the response speed difference indicates a difference between a first response speed of the first detection part and a second response speed of the second detection part.

2. The gas concentration detection device according to claim 1, wherein
the sensor element comprises the first element part and the second element part,
the gas concentration detection device further comprises a housing casing which comprises a male screw part to be engaged with an exhaust gas pipe through which the target gas flows,
the sensitivity correction part is configured to correct an error generated in the second detection part due to an angle difference between an upstream direction in a flow of the target gas and a surface direction of the outer surface of the second solid electrolyte body.

3. The gas concentration detection device according to claim 1, further comprising a deterioration detection part configured to detect occurrence of deterioration or a degree of deterioration of the second element part on the basis of a time difference between an initial time difference and either of a use time difference or a response speed difference between an initial response speed and a use response speed, wherein
the initial time difference represents a difference between a first output variation time detected by the first detection part and a second output variation time detected by the second detection part, obtained at an initial use of the gas concentration detection device, and the use time difference represents a difference between a first output variation time detected by the first detection part and a second output variation time detected by the second detection part, which is obtained after an elapse of a predetermined use time counted from the initial use of the gas concentration detection device,
the initial time difference represents a difference between a first response time of the first detection part and a second response time of the second detection part, obtained at the initial use of the gas concentration detection device, and the use time difference represents a difference between a first response time of the first detection part and a second response time of the second detection part, which is obtained after the elapse of the predetermined use time counted from the initial use of the gas concentration detection device, and
the response speed difference represents a difference between a first response time of the first detection part and a second response time of the second detection part, obtained at the initial use of the gas concentration detection device, and the use time difference represents a difference between a first response time of the first detection part and a second response time of the second detection part, which is obtained after the elapse of the predetermined use time counted from the initial use of the gas concentration detection device.

4. The gas concentration detection device according to claim 1, wherein
a pump electrode and a NOₓ electrode are formed on the surface of the first solid electrolyte body, to which the gas chamber is adjacently arranged, the gas chamber accommodates the pump electrode and the NOₓ electrode, the pump electrode adjusts an oxygen concentration in the target gas in the gas chamber, the NOₓ electrode detects a NOₓ concentration in the target gas in the gas chamber after the pump electrode has adjusted the oxygen concentration,
a reference gas duct and a first reference electrode are formed on the surface of the first solid electrolyte body, opposite in location to the surface adjacently arranged to the gas chamber, a reference gas is introduced into the reference gas duct accommodating the first reference electrode,
the pair of first electrodes are composed of the pump electrode and the first reference electrode,
the first detection part is configured to detect an oxygen concentration as a first gas component concentration,
a mixed potential electrode is formed on the outer surface of the second solid electrolyte body, the mixed potential electrode detects a mixed potential generated when an electrochemical reduction reaction of oxygen contained in the target gas, and an electrochemical oxidation of ammonia or nitrogen dioxide contained in the target gas are balanced,
a second reference electrode, accommodated in the reference gas duct, is formed on the surface of the second solid electrolyte body arranged adjacent to the reference gas duct,
the mixed potential electrode and the second reference electrode form the pair of second electrodes,
the second detection part is configured to detect an ammonia concentration or a nitrogen dioxide concentration as a second gas component concentration, and
the sensitivity correction part performs a correction by using a variation in concentration of oxygen as the common gas component which is varied by not less than the reference variation amount.

5. The gas concentration detection device according to claim 1, wherein
a pump electrode and a NOₓ electrode are formed on the surface of the first solid electrolyte body, to which the gas chamber is adjacently arranged, the gas chamber accommodates the pump electrode and the NOₓ electrode, the pump electrode adjusts an oxygen concentration in the target gas in the gas chamber, the NOₓ electrode detects a NOₓ concentration in the target gas in the gas chamber after the pump electrode has adjusted the oxygen concentration,
a reference gas duct and a first reference electrode are formed on the surface of the first solid electrolyte body, opposite in location to the surface adjacently arranged to the gas chamber, and a reference gas is introduced into the reference gas duct accommodating the first reference electrode,
the pair of first electrodes are composed of the NOₓ electrode and the first reference electrode,
the first detection part is configured to detect a NOₓ concentration as a first gas component concentration,
a mixed potential electrode is formed on the outer surface of the second solid electrolyte body, the mixed potential electrode detects a mixed potential generated when an electrochemical reduction reaction of oxygen contained in the target gas and an electrochemical oxidation of ammonia or nitrogen dioxide contained in the target gas are balanced,
a second reference electrode, accommodated in the reference gas duct, is formed on the surface of the second solid electrolyte body arranged adjacent to the reference gas duct,
the mixed potential electrode and the second reference electrode form the pair of second electrodes,
the second detection part is configured to detect an ammonia concentration or a nitrogen dioxide concentration as a second gas component concentration, and
the sensitivity correction part performs a correction based on a variation in concentration of ammonia or nitrogen dioxide as the common gas component which varies by not less than the reference variation amount.

6. The gas concentration detection device according to claim 1, wherein
a pump electrode is formed on the surface of the first solid electrolyte body, to which the gas chamber is adjacently arranged, the gas chamber accommodates the pump electrode, and the pump electrode adjusts an oxygen concentration in the target gas in the gas chamber,
a reference gas duct and a first reference electrode are formed on the surface of the first solid electrolyte body, opposite in location to the surface adjacently arranged to the gas chamber, a reference gas is introduced into the reference gas duct accommodating the first reference electrode,
the pair of first electrodes are composed of the pump electrode and the first reference electrode,
the first detection part is configured to detect an oxygen concentration as a first gas component concentration,
a mixed potential electrode is formed on the outer surface of the second solid electrolyte body, the mixed potential electrode detects a mixed potential generated when an electrochemical reduction reaction of oxygen contained in the target gas and an electrochemical oxidation of ammonia or nitrogen dioxide contained in the target gas are balanced,
a second reference electrode, accommodated in the reference gas duct, is formed on the surface of the second solid electrolyte body arranged adjacent to the reference gas duct,
the mixed potential electrode and the second reference electrode form the pair of second electrodes,
the second detection part is configured to detect an ammonia concentration or a nitrogen dioxide concentration as a second gas component concentration, and
the sensitivity correction part performs a correction based on a variation in concentration of ammonia or nitrogen dioxide as the common gas component which varies by not less than the reference variation amount.

7. The gas concentration detection device according to claim 1, wherein
the gas concentration detection device is configured to be used in an exhaust gas pipe of an internal combustion engine on which a reductant supply device is arranged, the reductant supply device supplies a reductant containing ammonia to a catalyst for reducing NOₓ, and
the sensitivity correction part performs correction by using a variation which is not less than a reference variation amount of oxygen as the common gas component at a start time or a completion time of a fuel cut operation of the internal combustion engine.

8. The gas concentration detection device according to claim 4, wherein
the gas concentration detection device is configured to be used in an exhaust gas pipe of an internal combustion engine on which a reductant supply device is arranged, the reductant supply device supplies a reductant containing ammonia to a catalyst for reducing NOₓ, and
the sensitivity correction part performs correction by using a variation which is not less than a reference variation amount of oxygen as the common gas component at a completion time of an idling operation of the internal combustion engine.

9. The gas concentration detection device according to claim 5, wherein
the gas concentration detection device is configured to be used in an exhaust gas pipe of an internal combustion engine on which a reductant supply device is arranged, the reductant supply device supplies a reductant containing ammonia to a catalyst for reducing NOₓ, and
the sensitivity correction part performs correction by using a variation which is not less than a reference variation amount of ammonia as the common gas component during a fuel cut operation or an idling operation of the internal combustion engine.
